(19) United States
(12) Patent Application Publication
(10) Pub. No.: US 2023/0181589 A1
(43) Pub. Date: Jun. 15, 2023

(54) PRMT5 INHIBITORS FOR OCULAR THERAPY

(71) Applicant: THE TRUSTEES OF INDIANA UNIVERSITY, Bloomington, IN (US)

(72) Inventors: Tao LU, Carmel, IN (US); Timothy William CORSON, Fishers, IN (US); Matthew MARTIN, Indianapolis, IN (US); Anbukkarasi MUNIYANDI, Indianapolis, IN (US); Mengyao SUN, Zionsville, IN (US); Kamakshi SISHTLA, Indianapolis, IN (US); Aishat Abiola MOTOLANI, Bloomington, IN (US)

(21) Appl. No.: 18/061,219

(22) Filed: Dec. 2, 2022

Related U.S. Application Data
(60) Provisional application No. 63/285,709, filed on Dec. 3, 2021.

(51) Int. Cl. A61K 31/519; A61P 27/02; A61K 45/06

(52) U.S. Cl. CPC A61K 31/519; A61K 45/06; A61P 27/02

(57) ABSTRACT

Methods and compositions to prevent or reduce the effects of neovascular eye disease in a subject are provided, wherein the subject is administered a pharmaceutical composition comprising an inhibitor of protein arginine methyltransferase 5 (PRMT5).

### CROSS-REFERENCE TO RELATED APPLICATIONS

This patent application claims the benefit of U.S. Provisional Pat. Application No. 63/285,709, filed Dec. 3, 2021, the entire disclosure of which is incorporated herein by reference.

### GOVERNMENT LICENSE RIGHTS

This invention was made with government support TR002529 awarded by the National Institutes of Health. The Government has certain rights in the invention.

### FIELD OF INVENTION

The present disclosure relates to methods and compositions to prevent or reduce the effects of neovascular eye disease in a subject are provided, wherein the subject is administered a pharmaceutical composition comprising an inhibitor of protein arginine methyltransferase 5 (PRMT5).

### BACKGROUND

Aberrant macular neovascularization is a hallmark of neovascular age-related macular degeneration (nvAMD). With approximately 20 million patients worldwide, this disease is one of the most common causes of blindness in older adults. The last two decades have seen an explosion of studies elucidating pathways involved in angiogenesis and the advent of effective antiangiogenic therapies primarily targeting the vascular endothelial growth factor (VEGF) signaling cascade. However, despite these successes over 30% of nvAMD patients are refractive to anti-VEGF therapy. Thus, there remains an urgent, unmet need to identify novel antiangiogenic targets leading to more effective therapies, not just for nvAMD, but for the range of other eye diseases characterized by neovascularization.

As disclosed herein applicant has identified protein arginine methyltransferase 5 (PRMT5) as a novel angiogenesis regulator. PRMT5 is a methyltransferase best known for regulating histone modifications. However, applicant has discovered that PRMT5 also methylates and activates NF-κB, a major proangiogenic and proinflammatory transcription factor. Accordingly, the PRMT5 enzyme is an appealing target for nvAMD therapeutic development.

### SUMMARY

Aberrant neovascularization is a hallmark of neovascular age-related macular degeneration (nvAMD) and other ocular diseases. Applicant has demonstrated that PRMT5 expression is increased during laser-induced choroidal neovascularization (L-CNV) in mice (this model recapitulates features of nvAMD). Moreover, PRMT5 inhibition with a novel, non-toxic small molecule inhibitor has been demonstrated herein to inhibit human retinal endothelial cell (HREC) and choroidal endothelial cell proliferation, migration, and tube formation in vitro without causing apoptosis, providing evidence of direct effects on cell types relevant to ocular neovascularization. Thus, PRMT5 is identified as a novel, druggable antiangiogenic target for ocular neovascular disease therapy. It is also demonstrated that inhibitors of PRS-LL-CM01 can inhibit the progression of neovascularization.

In accordance with one embodiment a method of treating neovascular eye disease is provided wherein the method comprising administering a PRMT5 inhibitor selected from the group consisting of compounds of formula (I), formula (II), formula (III), and formula (IV)

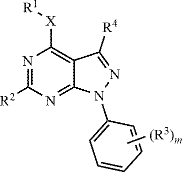

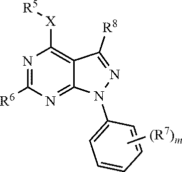

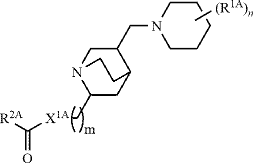

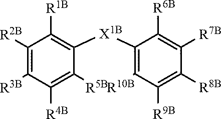

wherein
R¹ is hydrogen, halo, C₁-C₈ alkyl, C₃-C₆ cycloalkyl, heterocycloalkyl, aryl, or heteroaryl;
R² is C₁-C₈ alkyl, C₂-C₈ alkenyl, C₃-C₆ cycloalkyl, C₃-C₆ cycloalkylalkyl, heterocycloalkyl, aryl, arylalkyl, heteroaryl, C₁-C₈ alkoxy, C₃-C₆ cycloalkyloxy, aryloxy, halo, C₁-C₈ haloalkoxy, C₁-C₈ haloalkyl, haloaryl, haloaryloxy, —CN, —NO₂, --(CH₂)ₙC(O)R⁵, —(CH₂)ₙCO₂R⁵, —(CH₂)ₙC(O)NR⁵R⁶, —(CH₂)ₙNR⁵R⁶
—NH(CH₂)_{q}(N)CH₃CH₃, or —(CH₂)ₙNR⁵C(O)R⁶;
R³ is H, hydroxy, C₁-C₈ alkyl, C₂-C₈ alkenyl, C₃-C₆ cycloalkyl, C₃-C₆ cycloalkylalkyl, C₁-C₈ alkoxy, C₃-C₆ cycloalkyloxy, heterocycloalkyl, aryl, arylalkyl, heteroaryl, aryloxy, halo, C₁-C₈ haloalkyl, C₁-C₈ haloalkoxy, haloaryl, haloaryloxy, —CN, —NO₂, —C(O)R⁵, —CO₂R⁵, —C(O)NR⁵R⁶, —NR⁵C(O)R⁶, —(CH₂)ₙNR⁵R⁶, —(CH₂)ₙS O₂NR⁵R⁶, —(CH₂)ₙSO₂R⁵, aryl; or
two R³ moieties and the phenyl group to which they are attached form a naphthyl group that is optionally substituted;
R⁴ is H, hydroxy, C₁-C₈ alkyl, C₂-C₈ alkenyl, C₃-C₆ cycloalkyl, C₁-C₈ haloalkyl, —CN, —NO₂, —(CH₂)ₙNR⁵R⁶, heterocycloalkyl, aryl, or heteroaryl;
X is a bond, —(CH₂)ₒCR⁵R⁶—, —CR⁵R⁶(CH₂)ₒ—, —(CH₂)ₒNR⁵—, —NR⁵(CH₂)ₒ—, —(CH₂)ₒO—, or —O(CH₂)ₒ—,
R⁵ and R⁶ are the same or different and each is H or C₁-C₈ alkyl;
m, n, q and o are the same or different and each is 0 or an integer from 1-5, or a pharmaceutically acceptable salt thereof to a person afflicted with wherein a subject afflicted with neovascular eye disease.

In one embodiment a method of inhibit the progression of neovascular eye disease is provided wherein a subject afflicted with neovascular eye disease is administered a compound having a structure selected from the group consisting of:

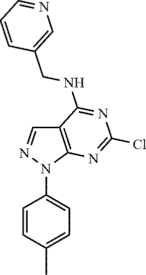 [SMILES: Cc1ccc(-n2ncc3c(NCc4cccnc4)nc(Cl)nc32)cc1]

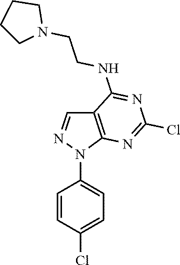 [SMILES: Clc1ccc(-n2ncc3c(NCCN4CCCC4)nc(Cl)nc32)cc1]

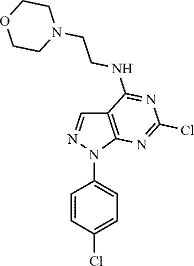 [SMILES: Clc1ccc(-n2ncc3c(NCCN4CCOCC4)nc(Cl)nc32)cc1]

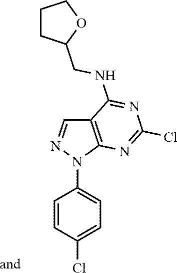

or a pharmaceutically acceptable salt thereof, optionally wherein the compound has the structure:

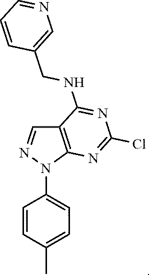

In one embodiment a method of inhibit the progression of neovascular eye disease is provided wherein a subject afflicted with neovascular eye disease is administered a compound of formula (I):

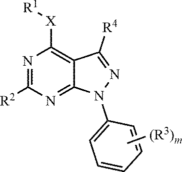

wherein
R¹ is selected from the group consisting of optionally substituted phenyl, piperazinyl, pyrrolyl, pyrrolidinyl pyranyl, piperidyl, morpholinyl, pyridinyl, and tetrahydrofuranyl;
R² is halo or —NH(CH₂)_{q}(N)CH₃CH₃;
R³ is H, C₁-C₈ alkyl or halo;
R⁴ is H;
X is —NH(CH₂)ₒ—;
o is 0, 1 or 2;
m is 0 or 1; and
q is 1 or 2, or a pharmaceutically acceptable salt thereof. In one embodiment a compound of Formula I is provided wherein
R¹ is a substituted or di-substituted phenyl, wherein the phenyl substituents are independently selected from C₁-C₄ alkyl;
R² is halo or —NH(CH₂)_{q}(N)CH₃CH₃;
R³ is H or halo;
R⁴ is H;
X is —NH(CH₂)ₒ—;
o is 0, 1 or 2; and q is 2, optionally wherein the compound has the structure of

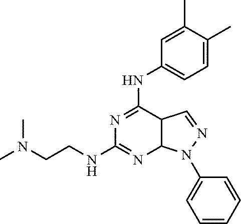 [SMILES: Cc1ccc(NC2=NC(NCCN(C)C)=NC3C2C=NN3c2ccccc2)cc1C]

In accordance with one embodiment compositions are provided for treating eye disease, particularly neovascular eye disease, wherein the compositions comprise any of the PRMT5 inhibitors disclosed herein and a pharmaceutically acceptable carrier. The compositions can further comprise other ocular therapeutic agents, including for example, a vascular endothelial growth factor (VEGF)/ vascular endothelial growth factor receptor (VEGFR) inhibitor. In accordance with one embodiment the PRMT5 inhibitor is an antisense RNA, shRNA, siRNA, RNA silencing or RNA interference (RNAi) targeting PRMT5 RNA; or a CRISPR/Cas9-mediated genetic ablation of PRMT5 genomic DNA, zinc-finger nuclease-mediated genetic ablation of PRMT5 genomic DNA, or any combination of PRMT5 inhibitors as disclosed herein.

In another aspect, the present disclosure is directed to a method of inhibiting choroidal neovascularization in a subject in need thereof, the method comprising administering to the subject a VEGF inhibitor, optionally in conjunction with the administration of any of the PRMT5 inhibitors disclosed herein.

In another aspect, the present disclosure is directed to a method of treating an ocular neovascular disease in a subject, the method comprising administering to the subject a VEGF inhibitor, optionally in conjunction with the administration of any of the PRMT5 inhibitors disclosed herein.

It has been found that inhibitors of VEGF can be used for treatments of various diseases, and particularly, for treatments of ocular neovascular diseases. In accordance with one embodiment any of the compositions comprising a PRMT5 inhibitor as disclosed herein can be combined with one or more vascular endothelial growth factor (VEGF)/ vascular endothelial growth factor receptor (VEGFR) inhibitors. Suitable VEGF inhibitors include, for example, pegaptanib, ranibizumab, aflibercept, bevacizumab, brolucizumab (also known as ESBA1008 and RTH258), conbercept (also known as KH-902), abicipar pegol, regorafenib, PAN-90806, Votrient (Generic name: pazopanib), Sutent (Generic name: sunitinib), Avastin (Generic name: bevacizumab), Nexavar (Generic name: sorafenib), Stivarga (Generic name: regorafenib), Cabometyx (Generic name: cabozantinib), Lenvima (Generic name: lenvatinib), Iclusig (Generic name: ponatinib), Cometriq (Generic name: cabozantinib), Zaltrap (Generic name: ziv-aflibercept), Inlyta (Generic name: axitinib), Zirabev (Generic name: bevacizumab), Mvasi (Generic name: bevacizumab), Fotivda (Generic name: tivozanib), Cyramza (Generic name: ramucirumab), and Caprelsa (Generic name: vandetanib).

Pharmaceutically acceptable carriers may be, for example, excipients, vehicles, diluents, and combinations thereof. For example, where the compositions are to be administered orally, they may be formulated as tablets, capsules, granules, powders, or syrups; or for parenteral administration, they may be formulated as injections (intramuscular, subcutaneous, intramedullary, intrathecal, intraventricular, intravenous, intravitreal), drop infusion preparations, or suppositories. For application by the ophthalmic mucous membrane route, they may be formulated as eye drops or eye ointments. These compositions can be prepared by conventional means, and, if desired, the active compound (i.e., PRMT5 inhibitor) may be mixed with any conventional additive, such as an excipient, a binder, a disintegrating agent, a lubricant, a corrigent, a solubilizing agent, a suspension aid, an emulsifying agent, a coating agent, or combinations thereof.

Suitable dosages of the PRMT5 inhibitors for use in the methods of the present disclosure will depend upon a number of factors including, for example, age and weight of an individual, severity of ocular neovascular disease, specific PRMT5 inhibitor to be used, nature of a composition, route of administration and combinations thereof. Ultimately, a suitable dosage can be readily determined by one skilled in the art such as, for example, a physician, a veterinarian, a scientist, and other medical and research professionals. For example, one skilled in the art can begin with a low dosage that can be increased until reaching the desired treatment outcome or result. Alternatively, one skilled in the art can begin with a high dosage that can be decreased until reaching a minimum dosage needed to achieve the desired treatment outcome or result. Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A method of treating eye disease in a subject, said method comprising administering to the subject a therapeutically effective amount of an inhibitor of protein arginine methyltransferase 5 (PRMT5).

2. The method of clause 1, wherein the eye disease is a neovascular eye disease.

3. The method of clause 1 or 2, wherein the eye disease is retinopathy of prematurity (ROP), proliferative diabetic retinopathy (PDR), wet age-related macular degeneration (AMD), neovascular age-related macular degeneration (nvAMD), pathological myopia, hypertensive retinopathy, occlusive vasculitis, polypoidal choroidal vasculopathy, diabetic macular edema, uveitic macular edema, central retinal vein occlusion, branch retinal vein occlusion, corneal neovascularization, retinal neovascularization, ocular histoplasmosis, neovascular glaucoma, or retinoblastoma.

4. The method of any one of the preceding clauses, wherein the eye disease is neovascular age-related macular degeneration (nvAMD).

5. The method of any one of the preceding clauses, wherein the inhibitor decreases the amount of active PRMT5 present in the eye of said subject.

6. The method of any one of the preceding clauses, wherein the method comprises administering to a subject a PRMT5 inhibitor selected from the group consisting of compounds of formula (I), formula (II), formula (III), and formula (IV)

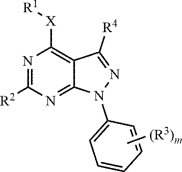

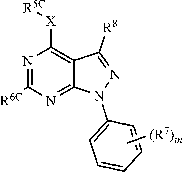

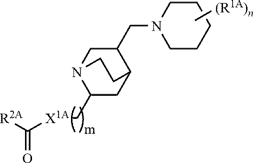

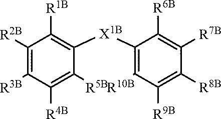

wherein
R¹ is hydrogen, halo, C₁-C₈ alkyl, C₃-C₆ cycloalkyl, heterocycloalkyl, aryl, or heteroaryl;
R² is C₁-C₈ alkyl, C₂-C₈ alkenyl, C₃-C₆ cycloalkyl, C₃-C₆ cycloalkylalkyl, heterocycloalkyl, aryl, arylalkyl, heteroaryl, C₁-C₈ alkoxy, C₃-C₆ cycloalkyloxy, aryloxy, halo,
C₁-C₈ haloalkoxy, C₁-C₈ haloalkyl, haloaryl, haloaryloxy, —CN, —NO₂, —(CH₂)ₙC(O)R⁵, —(CH₂)ₙCO₂R⁵, —(CH₂)ₙC(O)NR⁵R⁶, —(CH₂)ₙNR⁵R⁶
—NH(CH₂)_{q}(N)CH₃CH₃, or —(CH₂)ₙNR⁵C(O)R⁶;
R³ is H, hydroxy, C₁-C₈ alkyl, C₂-C₈ alkenyl, C₃-C₆ cycloalkyl, C₃-C₆ cycloalkylalkyl, C₁-C₈ alkoxy, C₃-C₆ cycloalkyloxy, heterocycloalkyl, aryl, arylalkyl, heteroaryl, aryloxy, halo, C₁-C₈ haloalkyl, C₁-C₈ haloalkoxy, haloaryl, haloaryloxy, —CN, —NO₂, —C(O)R⁵, —CO₂R⁵, —C(O)NR⁵R⁶, —NR⁵C(O)R⁶, —(CH₂)ₙNR⁵R⁶, —(CH₂)ₙSO₂NR⁵R⁶, —(CH₂)ₙSO₂R⁵, aryl; or
two R³ moieties and the phenyl group to which they are attached form a naphthyl group that is optionally substituted;
R⁴ is H, hydroxy, C₁-C₈ alkyl, C₂-C₈ alkenyl, C₃-C₆ cycloalkyl, C₁-C₈ haloalkyl, —CN, —NO₂, —(CH₂)ₙNR⁵R⁶, heterocycloalkyl, aryl, or heteroaryl;
X is a bond, —(CH₂)ₒCR⁵R⁶—, —CR⁵R⁶(CH₂)ₒ—, —(CH₂)ₒNR⁵—, —NR⁵(CH₂)ₒ—, —(CH₂)ₒO—, or —O(CH₂)ₒ—,
R⁵ and R⁶ are the same or different and each is H or C₁-C₈ alkyl;
m, n, q and o are the same or different and each is 0 or an integer from 1-5, or
a pharmaceutically acceptable salt thereof.

7. The method of any one of the preceding clauses , wherein the PRMT5 inhibitor is a compound selected from

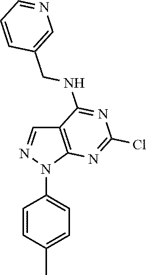 [SMILES: Cc1ccc(-n2ncc3c(NCc4cccnc4)nc(Cl)nc32)cc1]

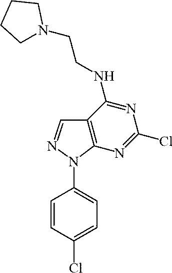 [SMILES: Clc1ccc(-n2ncc3c(NCCN4CCCC4)nc(Cl)nc32)cc1]

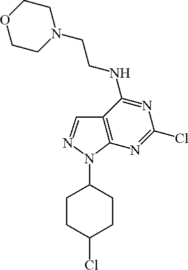 [SMILES: Clc1nc(NCCN2CCOCC2)c2cnn(C3CCC(Cl)CC3)c2n1]

and

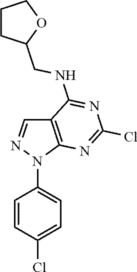 [SMILES: Clc1ccc(-n2ncc3c(NCC4CCCO4)nc(Cl)nc32)cc1]

or a pharmaceutically acceptable salt thereof.

8. The method of any one of clauses 1 to 6, wherein the PRMT5 inhibitor is a compound of formula (I):

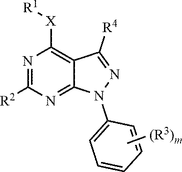

wherein
R¹ is selected from the group consisting of optionally substituted phenyl, piperazinyl, pyrrolyl, pyrrolidinyl pyranyl, piperidyl, morpholinyl, pyridinyl, and tetrahydrofuranyl;
R² is halo, —(CH₂)ₒCR⁵R⁶—, —(CH₂)ₒNR⁵ or —NH(CH₂)_{q}(N)R⁵R⁶;
R³ is H, C₁-C₈ alkyl or halo;
R⁴ is H or halo;
R⁵ and R⁶ are independently H or C₁-C₄ alkyl;
X is —NH(CH₂)ₒ—;
   is 0, 1 or 2;
m is 0 or 1; and
q is 1 or 2, or a pharmaceutically acceptable salt thereof.

9. The method of clause 8, wherein the PRMT5 inhibitor is a compound of Formula I wherein
R¹ is a substituted or di-substituted phenyl, wherein the phenyl substituents are independently selected from C₁-C₄ alkyl;
R² is halo or —NH(CH₂)_{q}(N)CH₃CH₃;
R³ is H or halo;
R⁴ is H;
X is —NH(CH₂)ₒ—;
   is 0, 1 or 2; m is 1, and q is 2, or a pharmaceutically acceptable salt thereof.

10. The method of any one of clauses 1 to 6, wherein the PRMT5 inhibitor has the structure of

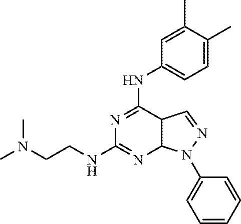 [SMILES: Cc1ccc(NC2=NC(NCCN(C)C)=NC3C2C=NN3c2ccccc2)cc1C]

or a pharmaceutically acceptable salt thereof.

11. The method of any one of the preceding clauses, wherein the PRMT5 inhibitor is administered via eye drops, eye ointment, or any combination thereof.

12. The method of any one of clauses 1-10 wherein the PRMT5 inhibitor is administered via intravitreal injection.

13. The method of any one of clauses 1-10 wherein the PRMT5 inhibitor is administered orally.

14. The method of any one of clauses 1 to 5, wherein the PRMT5 inhibitor is selected from antisense RNA; shRNA; siRNA; RNA silencing; RNA interference (RNAi) targeting PRMT5 RNA; CRISPR/Cas9-mediated genetic ablation of PRMT5 genomic DNA; zinc-finger nuclease-mediated genetic ablation of PRMT5 genomic DNA; and combinations thereof.

15. The method of any one of the preceding clauses, further comprising administering to the subject a therapeutically effective amount of a VEGF inhibitor.

16. The method of clauses 15, wherein the VEGF inhibitor is selected from the group comprising pegaptanib, ranibizumab, aflibercept, bevacizumab, brolucizumab (also known as ESBA1008 and RTH258), conbercept (also known as KH-902), abicipar pegol, regorafenib, PAN-90806, Votrient (Generic name: pazopanib), Sutent (Generic name: sunitinib), Avastin (Generic name: bevacizumab), Nexavar (Generic name: sorafenib), Stivarga (Generic name: regorafenib), Cabometyx (Generic name: cabozantinib), Lenvima (Generic name: lenvatinib), Iclusig (Generic name: ponatinib), Cometriq (Generic name: cabozantinib), Zaltrap (Generic name: ziv-aflibercept), Inlyta (Generic name: axitinib), Zirabev (Generic name: bevacizumab), Mvasi (Generic name: bevacizumab), Fotivda (Generic name: tivozanib), Cyramza (Generic name: ramucirumab), and Caprelsa (Generic name: vandetanib).

17. A method of inhibiting retinal angiogenesis, said method comprising contacting retinal endothelial cells with an inhibitor of protein arginine methyltransferase 5 (PRMT5).

18. The method of clause 17, said method comprising contacting retinal endothelial cells with a PRMT5 inhibitor having a structure selected from the group consisting of: formula (I), formula (II), formula (III), and formula (IV)

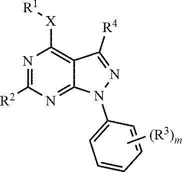

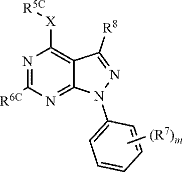

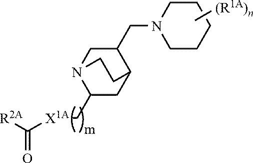

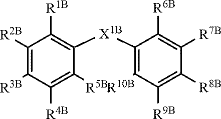

wherein
R¹ is hydrogen, halo, C₁-C₈ alkyl, C₃-C₆ cycloalkyl, heterocycloalkyl, aryl, or heteroaryl;
R² is C₁-C₈ alkyl, C₂-C₈ alkenyl, C₃-C₆ cycloalkyl, C₃-C₆ cycloalkylalkyl, heterocycloalkyl, aryl, arylalkyl, heteroaryl, C₁-C₈ alkoxy, C₃-C₆ cycloalkyloxy, aryloxy, halo, C₁-C₈ haloalkoxy, C₁-C₈ haloalkyl, haloaryl, haloaryloxy, —CN, —NO₂, —(CH₂)ₙC(O)R⁵, —(CH₂)ₙCO₂R⁵, —(CH₂)ₙC(O)NR⁵R⁶, —(CH₂)ₙNR⁵R⁶
—NH(CH₂)_{q}(N)CH₃CH₃, or —(CH₂)ₙNR⁵C(O)R⁶;
R³ is H, hydroxy, C₁-C₈ alkyl, C₂-C₈ alkenyl, C₃-C₆ cycloalkyl, C₃-C₆ cycloalkylalkyl, C₁-C₈ alkoxy, C₃-C₆ cycloalkyloxy, heterocycloalkyl, aryl, arylalkyl, heteroaryl, aryloxy, halo, C₁-C₈ haloalkyl, C₁-C₈ haloalkoxy, haloaryl, haloaryloxy, —CN, —NO₂, —C(O)R⁵, —CO₂R⁵, —C(O)NR⁵R⁶, —NR⁵C(O)R⁶, —(CH₂)ₙNR⁵R⁶, —(CH₂)ₙS O₂NR⁵R⁶, —(CH₂)ₙSO₂R⁵, aryl; or
two R³ moieties and the phenyl group to which they are attached form a naphthyl group that is optionally substituted;
R⁴ is H, hydroxy, C₁-C₈ alkyl, C₂-C₈ alkenyl, C₃-C₆ cycloalkyl, C₁-C₈
haloalkyl, —CN, —NO₂, —(CH₂)ₙNR⁵R⁶, heterocycloalkyl, aryl, or heteroaryl;
X is a bond, —(CH₂)ₒCR⁵R⁶—, —CR⁵R⁶(CH₂)ₒ—, —(CH₂)ₒNR⁵—, —NR⁵(CH₂)ₒ—, —(CH₂)ₒO—, or —O(CH₂)ₒ—,
R⁵ and R⁶ are the same or different and each is H or C₁-C₈ alkyl;
m, n, q and o are the same or different and each is 0 or an integer from 1-5, or
a pharmaceutically acceptable salt thereof.

19. The method of clauses 17 or 18, wherein the PRMT5 inhibitor is a compound selected from

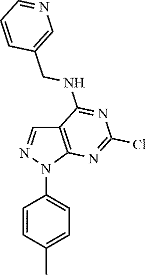 [SMILES: Cc1ccc(-n2ncc3c(NCc4cccnc4)nc(Cl)nc32)cc1]

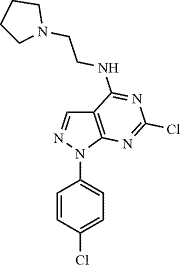 [SMILES: Clc1ccc(-n2ncc3c(NCCN4CCCC4)nc(Cl)nc32)cc1]

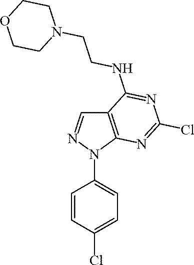 [SMILES: Clc1ccc(-n2ncc3c(NCCN4CCOCC4)nc(Cl)nc32)cc1]

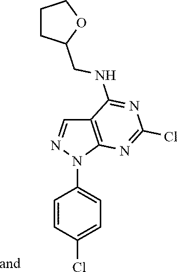

or a pharmaceutically acceptable salt thereof.

20. The method of any one of clauses 17 to 19, wherein the PRMT5 inhibitor is a compound of formula (I):

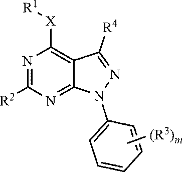

wherein
R¹ is selected from the group consisting of optionally substituted phenyl, piperazinyl, pyrrolyl, pyrrolidinyl pyranyl, piperidyl, morpholinyl, pyridinyl, and tetrahydrofuranyl;
R² is halo, —(CH₂)ₒCR⁵R⁶—, —(CH₂)ₒNR⁵ or —NH(CH₂)_{q}(N)R⁵R⁶;
R³ is H, C₁-C₈ alkyl or halo;
R⁴ is H or halo;
R⁵ and R⁶ are independently H or C₁-C₄ alkyl;
X is —NH(CH₂)ₒ—;
o is 0, 1 or 2;
m is 0 or 1; and
q is 1 or 2, or a pharmaceutically acceptable salt thereof.

21. The method of any one of clauses 17 to 20, wherein the PRMT5 inhibitor is a compound of Formula I wherein
R¹ is a substituted or di-substituted phenyl, wherein the phenyl substituents are independently selected from C₁-C₄ alkyl;
R² is halo or —NH(CH₂)_{q}(N)CH₃CH₃;
R³ is H or halo;
R⁴ is H;
X is —NH(CH₂)ₒ—;
o is 0, 1 or 2; and q is 2, or a pharmaceutically acceptable salt thereof.

22. The method of any one of clauses 17 to 21, wherein the PRMT5 inhibitor has the structure of

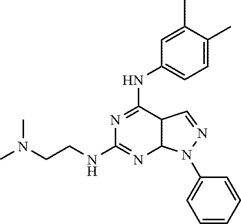 [SMILES: Cc1ccc(NC2=NC(NCCN(C)C)=NC3C2C=NN3c2ccccc2)cc1C]

or a pharmaceutically acceptable salt thereof.

23. The method of any one of clauses 17 to 22, wherein the PRMT5 inhibitor is administered via eye drops, eye ointment, or any combination thereof.

24. The method of any one of clauses 17 to 22, wherein the PRMT5 inhibitor is administered via intravitreal injection.

25. The method of any one of clauses 17 to 22, wherein the PRMT5 inhibitor is administered orally.

26. The method of clause 17, wherein the PRMT5 inhibitor is selected from antisense RNA; shRNA; siRNA; RNA silencing; RNA interference (RNAi) targeting PRMT5 RNA; CRISPR/Cas9-mediated genetic ablation of PRMT5 genomic DNA; zinc-finger nuclease-mediated genetic ablation of PRMT5 genomic DNA; and combinations thereof.

27. The method of any one of clauses 17 to 26, further comprising administering a VEGF inhibitor.

28. The method of clause 27, wherein the VEGF inhibitor is selected from the group comprising pegaptanib, ranibizumab, aflibercept, bevacizumab, brolucizumab (also known as ESBA1008 and RTH258), conbercept (also known as KH-902), abicipar pegol, regorafenib, PAN-90806, Votrient (Generic name: pazopanib), Sutent (Generic name: sunitinib), Avastin (Generic name: bevacizumab), Nexavar (Generic name: sorafenib), Stivarga (Generic name: regorafenib), Cabometyx (Generic name: cabozantinib), Lenvima (Generic name: lenvatinib), Iclusig (Generic name: ponatinib), Cometriq (Generic name: cabozantinib), Zaltrap (Generic name: ziv-aflibercept), Inlyta (Generic name: axitinib), Zirabev (Generic name: bevacizumab), Mvasi (Generic name: bevacizumab), Fotivda (Generic name: tivozanib), Cyramza (Generic name: ramucirumab), and Caprelsa (Generic name: vandetanib).

29. A method of inhibiting choroidal angiogenesis, said method comprising contacting choroidal endothelial cells with an inhibitor of protein arginine methyltransferase 5 (PRMT5).

30. The method of clause 29, said method comprising contacting choroidal endothelial cells with a PRMT5 inhibitor having a structure selected from the group consisting of: formula (I), formula (II), formula (III), and formula (IV)

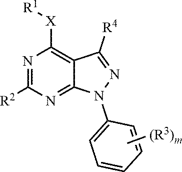

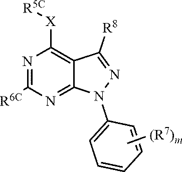

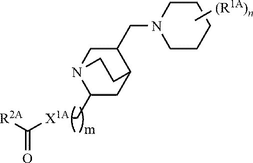

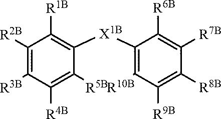

wherein
R¹ is hydrogen, halo, C₁-C₈ alkyl, C₃-C₆ cycloalkyl, heterocycloalkyl, aryl, or heteroaryl;
R² is C₁-C₈ alkyl, C₂-C₈ alkenyl, C₃-C₆ cycloalkyl, C₃-C₆ cycloalkylalkyl, heterocycloalkyl, aryl, arylalkyl, heteroaryl, C₁-C₈ alkoxy, C₃-C₆ cycloalkyloxy, aryloxy, halo, C₁-C₈ haloalkoxy, C₁-C₈ haloalkyl, haloaryl, haloaryloxy, —CN, —NO₂, —(CH₂)ₙC(O)R⁵, —(CH₂)ₙCO₂R⁵, —(CH₂)ₙC(O)NR⁵R⁶, —(CH₂)ₙNR⁵R⁶
—NH(CH₂)_{q}(N)CH₃CH₃, or —(CH₂)ₙNR⁵C(O)R⁶;
R³ is H, hydroxy, C₁-C₈ alkyl, C₂-C₈ alkenyl, C₃-C₆ cycloalkyl, C₃-C₆ cycloalkylalkyl, C₁-C₈ alkoxy, C₃-C₆ cycloalkyloxy, heterocycloalkyl, aryl, arylalkyl, heteroaryl, aryloxy, halo, C₁-C₈ haloalkyl, C₁-C₈ haloalkoxy, haloaryl, haloaryloxy, —CN, —NO₂, —C(O)R⁵, —CO₂R⁵, —C(O)NR⁵R⁶, —NR⁵C(O)R⁶, —(CH₂)ₙNR⁵R⁶, —(CH₂)ₙS O₂NR⁵R⁶, —(CH₂)ₙSO₂R⁵, aryl; or
two R³ moieties and the phenyl group to which they are attached form a naphthyl group that is optionally substituted;
R⁴ is H, hydroxy, C₁-C₈ alkyl, C₂-C₈ alkenyl, C₃-C₆ cycloalkyl, C₁-C₈ haloalkyl, —CN, —NO₂, —(CH₂)ₙNR⁵R⁶, heterocycloalkyl, aryl, or heteroaryl;
X is a bond, —(CH₂)ₒCR⁵R⁶—, —CR⁵R⁶(CH₂)ₒ—, —(CH₂)ₒNR⁵—, —NR⁵(CH₂)ₒ—, —(CH₂)ₒO—, or —O(CH₂)ₒ—,
R⁵ and R⁶ are the same or different and each is H or C₁-C₈ alkyl;
m, n, q and o are the same or different and each is 0 or an integer from 1-5, or
a pharmaceutically acceptable salt thereof.

31. The method of clause 29 or 30, wherein the PRMT5 inhibitor is a compound selected from

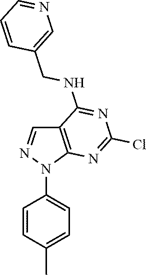 [SMILES: Cc1ccc(-n2ncc3c(NCc4cccnc4)nc(Cl)nc32)cc1]

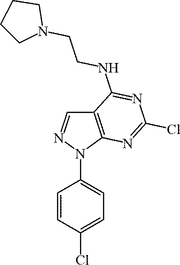 [SMILES: Clc1ccc(-n2ncc3c(NCCN4CCCC4)nc(Cl)nc32)cc1]

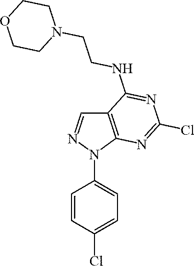 [SMILES: Clc1ccc(-n2ncc3c(NCCN4CCOCC4)nc(Cl)nc32)cc1]

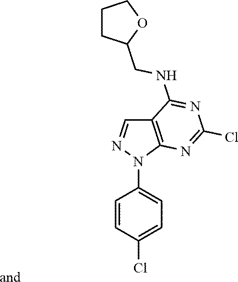

or a pharmaceutically acceptable salt thereof.

32. The method of any one of clauses 29 to 31, wherein the PRMT5 inhibitor is a compound of formula (I):

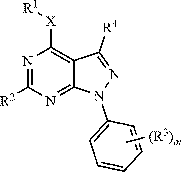

wherein
R¹ is selected from the group consisting of optionally substituted phenyl, piperazinyl, pyrrolyl, pyrrolidinyl pyranyl, piperidyl, morpholinyl, pyridinyl, and tetrahydrofuranyl;
R² is halo, —(CH₂)ₒCR⁵R⁶—, —(CH₂)ₒNR^{S} or —NH(CH₂)_{q}(N)R⁵R⁶;
R³ is H, C₁-C₈ alkyl or halo;
R⁴ is H or halo;
R⁵ and R⁶ are independently H or C₁-C₄ alkyl;
X is —NH(CH₂)ₒ—;
o is 0, 1 or 2;
m is 0 or 1; and
q is 1 or 2, or a pharmaceutically acceptable salt thereof.

33. The method of any one of clauses 29 to 32, wherein the PRMT5 inhibitor is a compound of Formula I wherein
R¹ is a substituted or di-substituted phenyl, wherein the phenyl substituents are independently selected from C₁-C₄ alkyl;
R² is halo or —NH(CH₂)_{q}(N)CH₃CH₃;
R³ is H or halo;
R⁴ is H;
X is —NH(CH₂)ₒ—;
o is 0, 1 or 2; and q is 2, or a pharmaceutically acceptable salt thereof.

34. The method of any one of clauses 29 to 33, wherein the PRMT5 inhibitor has the structure of

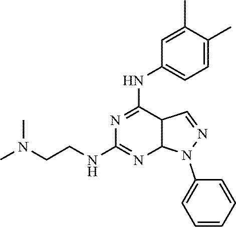 [SMILES: Cc1ccc(NC2=NC(NCCN(C)C)=NC3C2C=NN3c2ccccc2)cc1C]

or a pharmaceutically acceptable salt thereof.

35. The method of any one of clauses 29 to 34, comprising administering the PRMT5 inhibitor via eye drops, eye ointment, or any combination thereof.

36. The method of any one of clauses 29 to 34, comprising administering the PRMT5 inhibitor via intravitreal injection.

37. The method of any one of clauses 29 to 34, wherein the PRMT5 inhibitor is administered orally.

38. The method of clause 29, wherein the PRMT5 inhibitor is selected from antisense RNA; shRNA; siRNA; RNA silencing; RNA interference (RNAi) targeting PRMT5 RNA; CRISPR/Cas9-mediated genetic ablation of PRMT5 genomic DNA; zinc-finger nuclease-mediated genetic ablation of PRMT5 genomic DNA; and combinations thereof.

39. The method of any one any one of clauses 29 to 38, further comprising administering a VEGF inhibitor.

40. The method of clause 39, wherein the VEGF inhibitor is selected from the group comprising pegaptanib, ranibizumab, aflibercept, bevacizumab, brolucizumab (also known as ESBA1008 and RTH258), conbercept (also known as KH-902), abicipar pegol, regorafenib, PAN-90806, Votrient (Generic name: pazopanib), Sutent (Generic name: sunitinib), Avastin (Generic name: bevacizumab), Nexavar (Generic name: sorafenib), Stivarga (Generic name: regorafenib), Cabometyx (Generic name: cabozantinib), Lenvima (Generic name: lenvatinib), Iclusig (Generic name: ponatinib), Cometriq (Generic name: cabozantinib), Zaltrap (Generic name: ziv-aflibercept), Inlyta (Generic name: axitinib), Zirabev (Generic name: bevacizumab), Mvasi (Generic name: bevacizumab), Fotivda (Generic name: tivozanib), Cyramza (Generic name: ramucirumab), and Caprelsa (Generic name: vandetanib).

41. The method of any one of clauses 1-10, wherein the PRMT5 inhibitor is administered via eye drops, eye ointment, intravitreal injection, orally, or any combination thereof.

### BRIEF DESCRIPTION OF THE DRAWINGS

FIG. 1A is a graph showing the results of an in vitro proliferation assay in disease-relevant, primary human retinal microvascular endothelial cells (HRECs) showing the activity of compound PR5—LL—CM01. GI50 value is indicated. Mean ± S.E.M., n = 3 per dose.

FIG. 1B is a graph showing the results of an in vitro proliferation assay in disease-relevant, iPSC-derived choroidal endothelial cells (iCEC2) showing the activity of compound PR5—LL—CM01. GI50 value is indicated. Mean ± S.E.M., n = 3 per dose.

FIG. 2A is an image showing the results of PRMT5 immunostaining on sections of eyes from human nvAMD patient (68 year old, female), where DAPI (blue) shows the nuclei of the cells and magenta indicates PRMT5 expression in different layers of the retina, and in the RPE/choroid complex. Scale bars = 20 µm. GCL, ganglion cell layer; INL, inner nuclear layer; ONL, outer nuclear layer; IS/OS photoreceptor inner/outer segments; CC, choriocapillaris.

FIG. 2B is an image showing the results of PRMT5 immunostaining on sections of eyes from human control patient (81 year old female), where DAPI (blue) shows the nuclei of the cells and magenta indicates PRMT5 expression in different layers of the retina, and in the RPE/choroid complex. Scale bars = 20 µm.

FIG. 2C is an image showing the results of PRMT5 immunostaining on sections of eyes from preimmune IgG control, where DAPI (blue) shows the nuclei of the cells and magenta indicates PRMT5 expression in different layers of the retina, and in the RPE/choroid complex. Scale bars = 20 µm.

FIG. 2D is a graph showing the quantification of PRMT5 mean fluorescence intensity (MFI) in RPE and retina of nvAMD vs controls. Mean±SEM, n=3. *p<0.05, Student’s t-test with Welch’s correction.

FIG. 3A is an image showing the PRMT5 expression results by flat mount staining of murine laser-induced choroidal neovascularization (L-CNV) choroids compared to untouched control. The expression of PRMT5 (magenta) is observed in and around the neovascular lesion in the choroid that underwent laser treatment.

FIG. 3B is an image showing the results of an immunoblot assay showing PRMT5 expression in the retina and choroid of the L-CNV mouse eyes and untouched control eyes.

FIG. 3C is an image showing the PRMT5 expression results in different layers of the retina by immunostaining of cryosections of the L-CNV retina. The expression of PRMT5 (magenta) is observed in and around the ganglion cell layer (GCL), inner plexiform layer (IPL), outer plexiform layer (OPL), and in the inner and outer segments (IS/OS) of photoreceptors, where neovascularization is observed (isolectin B4 [IB4] staining, green).

FIG. 3D is an image showing the PRMT5 expression results by immunostaining of cryosections of the L-CNV choroid. The expression of PRMT5 (magenta) is observed in the retinal pigment epithelium (RPE)-choroid complex, where neovascularization is observed (isolectin B4 [IB4] staining, green).

FIG. 4A is an image showing the results of an immunoblot assay showing overexpression of Flag-tagged WT-PRMT5 in HRECs.

FIG. 4B is an image showing the results of an immunoblot assay showing knocked down expression of PRMT5 in HRECs.

FIG. 4C is an image showing the results of an immunoblot assay showing overexpression of Flag-tagged WT-PRMT5 in iCEC2 cells. pLV-empty is a vector control.

FIG. 4D is an image showing the results of an immunoblot assay showing knocked down expression of PRMT5 in iCEC2 cells. shPRMT5 is shRNA knockdown of PRMT5, and shScramble is control.

FIG. 5A is a graph showing the results of a NF-κB luciferase assay in HREC cells showing inhibition of PRMT5 activity modulates NF-κB activity. Vector control (pLV-empty, left set) or WT-PRMT5 overexpressing cells (pLV-wtPRMT5, right set) were stimulated with 10 ng/ml IL-10 ± 3 µM PR5-LL-CM01 for 4 hours. *p<0.05 vs. IL-1β untreated group; #p<0.05 vs. IL-1β-induced group; $, p<0.05 vs. vector + IL-1β-treated group.

FIG. 5B is a graph showing the results of a NF-κB luciferase assay in iCEC2 cells showing inhibition of PRMT5 activity modulates NF-κB activity. Vector control (pLV-empty, left set) or WT-PRMT5 overexpressing cells (pLV-wtPRMT5, right set) were stimulated with 10 ng/ml IL-10 ± 3 µM PR5-LL-CM01 for 4 hours. *p<0.05 vs. IL-1β untreated group; #p<0.05 vs. IL-1β-induced group; $, p<0.05 vs. vector + IL-1β-treated group.

FIG. 5C is a graph showing the results of a NF-κB luciferase assay in HREC cells showing inhibition of PRMT5 activity modulates NF-κB activity. Both shScramble (shScram, left set) and shPRMT5 HRECs (right set) were triggered by IL-1β for 4 hours. *p<0.05 vs. IL-1β untreated group; $p<0.05 vs. shScram + IL-1β-treated group. N=3-4.

FIG. 5D is a graph showing the results of a NF-κB luciferase assay in iCEC2 cells showing inhibition of PRMT5 activity modulates NF-κB activity. Both shScramble (shScram, left set) and shPRMT5 iCEC2 (right set) were triggered by IL-1β for 4 hours. *p<0.05 vs. IL-1β untreated group; $p<0.05 vs. shScram + IL-1β-treated group. N=3-4.

FIG. 6A is a graph showing the results of a qPCR assay in HREC vector-transduced cells showing inhibition of NF-κB target gene tumor necrosis factor α (TNFα) by PR5-LL-CM01 or shPRMT5 knockdown. Cells were stimulated with 10 ng/ml IL-10 ± 3 µM PR5-LL-CM01 for 4 hours. *p<0.05 vs. IL-1β untreated and PR5-LL-CM01 untreated group; #p<0.05 vs. IL-1β-induced group, and PR5-LL-CM01 untreated group; $, p<0.05 vs. shScram group.

FIG. 6B is a graph showing the results of a qPCR assay in HREC vector-transduced cells showing inhibition of NF-κB target gene vascular endothelial growth factor (VEGFA) by PR5-LL-CM01 or shPRMT5 knockdown. Cells were stimulated with 10 ng/ml IL-1β ± 3 µM PR5-LL-CM01 for 4 hours. *p<0.05 vs. IL-1β untreated and PR5-LL-CM01 untreated group; #p<0.05 vs. IL-1β-induced group, and PR5-LL-CM01 untreated group; $, p<0.05 vs. shScram group.

FIG. 6C is a graph showing the results of a qPCR assay in iCEC2 vector-transduced cells showing inhibition of NF-κB target genes tumor necrosis factor α (TNFα) by PR5-LL-CM01 or shPRMT5 knockdown. Cells were stimulated with 10 ng/ml IL-10 ± 3 µM PR5-LL-CM01 for 4 hours. *p<0.05 vs. IL-1β untreated and PR5-LL-CM01 untreated group; #p<0.05 vs. IL-1β-induced group, and PR5-LL-CM01 untreated group; $, p<0.05 vs. shScram group.

FIG. 6D is a graph showing the results of a qPCR assay in iCEC2 vector-transduced cells showing inhibition of NF-κB target gene vascular endothelial growth factor (VEGFA) by PR5-LL-CM01 or shPRMT5 knockdown. Cells were stimulated with 10 ng/ml IL-1β ± 3 µM PR5-LL-CM01 for 4 hours. *p<0.05 vs. IL-1β untreated and PR5-LL-CM01 untreated group; #p<0.05 vs. IL-1β-induced group, and PR5-LL-CM01 untreated group; $, p<0.05 vs. shScram group.

FIG. 7A is a graph showing the results of PRMT5 on HREC cell proliferation showing overexpression of WT-PRMT5 (pLV-wtPRMT5, top curve) promoted cell growth compared to vector comotrol (pLV-empty, bottom curve). Mean±SEM, n=3-4 biological replicates; *p<0.05 WT-PRMT5

FIG. 7B is a graph showing the results of PRMT5 on HREC cell proliferation showing that using shRNA to knockdown PRMT5 (shPRMT5, bottom curve) does not promote cell growth compared to control (shScramble, top curve). Mean±SEM, n=3-4 biological replicates; *#p<0.05, shPRMT5 vs. shScram.

FIG. 7C is a graph showing the results of PRMT5 on iCEC2 cell proliferation showing overexpression of WT-PRMT5 (pLV-wtPRMT5, top curve) promoted cell growth compared to vector comotrol (pLV-empty, bottom curve). Mean±SEM, n=3-4 biological replicates; *p<0.05 WT-PRMT5

FIG. 7D is a graph showing the results of PRMT5 on iCEC2 cell proliferation showing that using shRNA to knockdown PRMT5 (shPRMT5, bottom curve) does not promote cell growth compared to control (shScramble, top curve). Mean±SEM, n=3-4 biological replicates; *#p<0.05, shPRMT5 vs. shScram

FIG. 8 is an image showing the results of a cell migration assay of HREC cells treated with indicated concentrations of PR5-LL-CM01. Cells migrated into the blue scratch area counted. Mean±SEM, N=6 images, two independent experiments. ***p<0.001, ANOVA with Dunnett’s post hoc test.

FIG. 9A is an image showing the results of a quantitative analysis of tube formation in HREC cells showing that PR5-LL-CM01 blocks tube formation in a dose-dependent manner. Mean±SEM, n=6-12 images. **p<0.01; ***p<0.001 vs. DMSO control, one-way ANOVA with Dunnett’s post hoc test. Representative data from three biological replicates. Scale bars = 500 µm.

FIG. 9B is an image showing the results of a quantitative analysis of tube formation in iCEC2 cells showing that PR5-LL-CM01 blocks tube formation in a dose-dependent manner. Mean±SEM, n=6-12 images. **p<0.01; ***p<0.001 vs. DMSO control, one-way ANOVA with Dunnett’s post hoc test. Representative data from three biological replicates. Scale bars = 500 µm.

FIG. 9C is an image showing the results of a quantitative analysis of tube formation in HREC cells transduced with shScramble vector or shPRMT5 showing that that knockdown of PRMT5 reduces tube formation ability. Mean±SEM, n=6-12 images. ***p<0.0001 vs. shScramble control, unpaired Student’s t-test with Welch’s correction. Scale bars = 500 µm.

FIG. 9D is an image showing the results of a quantitative analysis of tube formation in iCEC2 cells transduced with shScramble vector or shPRMT5 showing that that knockdown of PRMT5 reduces tube formation ability. Mean±SEM, n=6-12 images. ***p<0.0001 vs. shScramble control, unpaired Student’s t-test with Welch’s correction. Scale bars = 500 µm.

FIG. 10A is a graph of a cell cycle profile showing the results of a flow cytometry cell cycle assay of HREC cells showing PR5-LL-CM01 dose-dependently decreases cells in S-phase and increases cells in G0/G1 after 24 hours of treatment. Mean±SEM of percentage of cells, n=3 biological replicates.

FIG. 10B is a graph of a cell cycle profile showing the results of a flow cytometry cell cycle assay of iCEC2 cells showing PR5-LL-CM01 dose-dependently decreases cells in S-phase and increases cells in G0/G1 after 24 hours of treatment. Mean±SEM of percentage of cells, n=3 biological replicates.

FIG. 11 is a figure of a hypothetical model showing PR5-LL-CM01 inhibition of PRMT5. PRMT5 methylates and activates NF-κB, resulting in the induction of NF-κB downstream genes, including cytokines, angiogenesis factors, chemokines, and antiapoptotic genes.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination.

### Definitions

In describing and claiming the methods, the following terminology will be used in accordance with the definitions set forth below.

The term “about” as used herein means greater or lesser than the value or range of values stated by 10 percent, but is not intended to designate any value or range of values to only this broader definition. Each value or range of values preceded by the term “about” is also intended to encompass the embodiment of the stated absolute value or range of values.

As used herein, the term “alkyl” refers to a mono-valent saturated aliphatic chain of carbon atoms, which is optionally branched and contains from 1 to 20 carbon atoms. It is to be further understood that in certain embodiments, alkyl may be advantageously of limited length, including C₁-C₁₂, C₁-C₁₀, C₁-C₉, C₁-C₈, C₁-C₇, C₁-C₆, and C₁-C₄, Illustratively, such particularly limited length alkyl groups, including C₁-C₈, C₁-C₇, C₁-C₆, and C₁-C₄, and the like may be referred to as “lower alkyl.” Illustrative alkyl groups include, but are not limited to, methyl, ethyl, n-propyl, isopropyl, n-butyl, isobutyl, sec-butyl, tert-butyl, pentyl, 2-pentyl, 3-pentyl, neopentyl, and the like. Alkyl may be substituted as described herein or unsubstituted.

The term “cycloalkyl” refers to a saturated or partially saturated, monocyclic or polycyclic mono-valent carbocycle. In some embodiments, it can be advantageous to limit the number of atoms in a “cycloalkyl” to a specific range of atoms, such as having 3 to 12 ring atoms. Illustrative alkyl groups include, but are not limited to, cyclopropyl, cyclobutyl, cyclopentyl, cycloheptyl, cyclooctyl, cyclononyl, and cyclodecyl. Cycloalkyl may be substituted as described herein or unsubstituted.

The term “aryl” refers to a monovalent all-carbon monocyclic or fused-ring polycyclic group having a completely conjugated pi-electron system. In some embodiments, it can be advantageous to limit the number of atoms in an “aryl” to a specific range of atoms, such as all-carbon monocyclic or fused-ring polycyclic groups of 6 to 14 carbon atoms (C₆-C₁₄ aryl). Examples, without limitation, of aryl groups are phenyl, naphthalenyl and anthracenyl. Aryl may be substituted as described herein or unsubstituted.

The term “heterocycloalkyl” or “heterocyclyl” refers to a mono-valent monocyclic or polycyclic ring structure that is saturated or partially saturated having one or more non-carbon ring atoms. Illustrative examples of heterocycloalkyl groups include, but are not limited to, monovalent radicals of the following entities, tetrahydrofuranyl, morpholinyl, dioxanyl, pyrrolidinyl, and piperidinyl.

The term “heteroaryl” refers to a mono-valent monocyclic, fused bicyclic, or fused polycyclic aromatic heterocycle (ring structure having ring atoms or members selected from carbon atoms and up to four heteroatoms selected from nitrogen, oxygen, and sulfur) that is fully unsaturated and having from 3 to 12 ring atoms per heterocycle. Non-limiting examples of five-membered heteroaryl groups include mono-valent radicals of furan, thiophene, pyrrole, oxazole, isoxazole, thiazole, isothiazole, pyrazole, imidazole, oxadiazole, thiadiazole, triazole, or tetrazole. Non-limiting examples of six-membered heteroaryl groups include monovalent radicals of pyridine, pyrazine, pyrimidine, pyridazine, or triazine.

As used herein, the term “halo” refers to fluoro (-F), chloro (-Cl), bromo (-Br), or iodo (-I).

As used herein, the term “amine” refers to a functional group that contains a basic nitrogen atom with a lone pair. Amines are derivatives of ammonia (NH₃), wherein one or more hydrogen atoms have been replaced by a substituent. Illustratively, an amine group may be represented by the formula —(N)R⁵R⁶, wherein the nitrogen atom is bound to a preceding atom and two R groups. For example, an amine group as used herein can be represented by —(N)CH₃CH₃ or —N(CH₃)₂, or —NCH₃CH₃.

As used herein, the term “treating” includes alleviation of the symptoms associated with a specific disorder or condition and/or preventing or eliminating said symptoms.

As used herein the terms “effective amount” or “therapeutically effective amount” of a compound refers to a nontoxic but sufficient amount of the compound to provide the desired effect. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

As used herein, unless specifically provided to the contrary, % and wt. % will equally mean % (percent) by weight of the total weight.

The term “parenteral” means not through the alimentary canal but by some other route such as intranasal, intraocular, inhalation, subcutaneous, intramuscular, intraspinal, or intravenous.

As used herein, the term “purified” relates to the isolation of a molecule or compound in a form that is substantially free of contaminants normally associated with the molecule or compound in a native or natural environment and means having been increased in purity as a result of being separate from other components of the original composition.

As used herein, the terms “maintain,” “prevent,” and “reduce the effects of” means to not lose up to about 5% or gain up to about 5% of tissue mass.

As used herein, the term “subject” means an animal including but not limited to, humans, domesticated animals including horses, dogs, cats, cattle, and the like, rodents, reptiles, and amphibians

For use purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the disclosure are sterile compositions. Pharmaceutical compositions may be prepared using compounding techniques known or that become available to those skilled in the art.

As used herein the term “patient” or “subject” means an animal including but not limited to, humans, domesticated animals including horses, dogs, cats, cattle, and the like, rodents, reptiles, and amphibians being administered a therapeutic treatment either with or without physician oversight.

As used herein, the term “vascular endothelial growth factor (VEGF)/ vascular endothelial growth factor receptor (VEGFR) inhibitors” defines agents that inhibit the activity of VEGF and VEGFR. VEGR and VEGFR (a tyrosine kinase receptor) signaling modulates angiogenesis, which involves making new blood vessels from existing blood vessels.

The compounds described herein may be used in pharmaceutical compositions or methods in combination with one or more additional active ingredients (or therapeutic agent) in the treatment of the diseases and disorders described herein. Further additional active ingredients include other therapeutics or agents that mitigate adverse effects of therapies for the intended disease targets. Such combinations may serve to increase efficacy, ameliorate other disease symptoms, decrease one or more side effects, or decrease the required dose of an inventive compound. The additional active ingredients may be administered in a separate pharmaceutical composition from a compound of the present disclosure or may be included with a compound of the present disclosure in a single pharmaceutical composition. The additional active ingredients may be administered simultaneously with, prior to, or after administration of a compound of the present disclosure.

Combination agents include additional active ingredients or therapeutic agents that are known or discovered to be effective in treating the diseases and disorders described herein. For example, compositions and formulations of the disclosure, as well as methods of treatment, can further comprise other drugs or pharmaceuticals, e.g., other therapeutic agents useful for treating or palliative for the target diseases or related symptoms or conditions.

### Embodiments

As demonstrated herein, PRMT5 expression is increased during laser-induced choroidal neovascularization (L-CNV) in mice (this model recapitulates features of nvAMD). Moreover, PRMT5 inhibition with a novel, non-toxic small molecule inhibitor (PR5-LL-CM01 as described in U.S. Pat. #11034,689, the disclosure of which is expressly incorporated herein) inhibits human retinal endothelial cell (HREC) proliferation, migration, and tube formation in vitro without causing apoptosis, providing evidence of direct effects on a cell type relevant to ocular neovascularization. Applicant is the first to discover that PRMT5 is an antiangiogenic target for ocular neovascular disease therapy. Accordingly, in one embodiment the present invention is directed to the use of inhibitors of PRMT5 for inhibiting the progression of neovascular diseases such as nvAMD.

In accordance with one embodiment compounds of formulas (I) - (IV) having activity in inhibiting PRMT5 are used to inhibit human retinal endothelial cell (HREC) and/or choroidal endothelial cell proliferation, migration, and/or tube formation. Accordingly applicant has discovered that such PRMT5 inhibitors can be used in the treatment of ocular neovascular diseases, including nvAMD.

In one embodiment, a PRMT5 inhibitor is provided having the structure of formula (I)

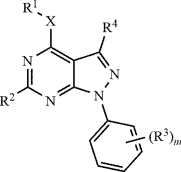

wherein
R¹ is hydrogen, halo, C₁-C₈ alkyl, C₃-C₆ cycloalkyl, heterocycloalkyl, aryl, substituted aryl or heteroaryl;
R² is C₁-C₈ alkyl, C₂-C₈ alkenyl, C₃-C₆ cycloalkyl, C₃-C₆ cycloalkylalkyl, heterocycloalkyl, aryl, arylalkyl, heteroaryl, C₁-C₈ alkoxy, C₃-C₆ cycloalkyloxy, aryloxy, halo, C₁-C₈ haloalkoxy, haloaryl, haloaryloxy, C₁-C₈ haloalkyl, —CN, —NO₂, —(CH₂)ₙC(O)R⁵,
—(CH₂)ₙCO₂R⁵, —(CH₂)ₙC(O)NR⁵R⁶, —(CH₂)ₙNR⁵C(O)R⁶, -NH(CH₂)q(N)CH₃CH₃ or —(CH₂)ₙNR⁵R⁶;
R³ is hydroxy, C₁-C₈ alkyl, C₂-C₈ alkenyl, C₃-C₆ cycloalkyl, C₃-C₆ cycloalkylalkyl, C₁-C₈ alkoxy, C₃-C₆ cycloalkyloxy, heterocycloalkyl, aryl, arylalkyl, heteroaryl, aryloxy, halo, C₁-C₈ haloalkyl, C₁-C₈ haloalkoxy, haloaryl, haloaryloxy, —CN, —NO₂, —C(O)R⁵, —CO₂R⁵, —C(O)NR⁵R⁶, —NR⁵C(O)R⁶, —(CH₂)ₙNR⁵R⁶, —(CH₂)ₙS O₂NR⁵R⁶, —(CH₂)ₙSO₂R⁵, aryl; or
two R³ moieties and the phenyl group to which they are attached form a naphthyl group that is optionally substituted;
R⁴ is H, hydroxy, C₁-C₈ alkyl, C₂-C₈ alkenyl, C₃-C₆ cycloalkyl, C₁-C₈ haloalkyl, —CN, —NO₂, —(CH₂)ₙNR⁵R⁶, heterocycloalkyl, aryl, or heteroaryl;
X is a bond, —(CH₂)ₒCR⁵R⁶—, —CR⁵R⁶(CH₂)ₒ—, —(CH₂)ₒNR⁵—, —NR⁵(CH₂)ₒ—, —(CH₂)ₒO—, or —O(CH₂)ₒ—,
R⁵ and R⁶ are the same or different and each is H or C₁-C₈ alkyl;
m, n, q and o are the same or different and each is 0 or an integer from 1-5, or
a pharmaceutically acceptable salt thereof.

In one embodiment the compound of formula (I) is provided wherein, R¹ is hydrogen, halo, C₁-C₈ alkyl, C₃-C₆ cycloalkyl, aryl, heteroaryl or heterocycloalkyl, optionally wherein R¹ is selected from the group consisting of phenyl, substituted phenyl, isoxazolyl, thiazolinyl, imidazolidinyl, piperazinyl, homopiperazinyl, pyrrolyl, pyrrolinyl, pyrazolyl, pyranyl, piperidyl, oxazolyl, and morpholinyl, pyridinyl, pyridazinyl, pyrimidyl, pyrazinyl, benzimidazolyl, triazinyl, imidazolyl, (1,2,3)-triazolyl, (1,2,4)-triazolyl, tetrazolyl, furyl, thienyl, isothiazolyl, thiazolyl, isoxazolyl, and oxadiazolyl. Preferably, R¹ is halo, heterocycloalkyl selected from the group consisting of isoxazolyl, thiazolinyl, imidazolidinyl, piperazinyl, homopiperazinyl, pyrrolyl, pyrrolinyl, pyrazolyl, pyranyl, piperidyl, oxazolyl, and morpholinyl, or heteroaryl selected from the group consisting of phenyl, substituted phenyl, pyridinyl, pyridazinyl, pyrimidyl, pyrazinyl, benzimidazolyl, triazinyl, imidazolyl, (1,2,3)-triazolyl, (1,2,4)-triazolyl, pyrazinyl, tetrazolyl, furyl, pyrrolyl, thienyl, isothiazolyl, thiazolyl, isoxazolyl, and oxadiazolyl.

In any of the foregoing embodiments of the compound of formula (I), R² is halo, C₁-C₈ haloalkoxy, C₁-C₈ haloalkyl, haloaryl, or haloaryloxy.

In any of the foregoing embodiments of the compound of formula (I), R³ is C₁-C₈ alkyl, halo, or C₁-C₈ haloalkyl.

In any of the foregoing embodiments of the compound of formula (I), R⁴ is H.

In any of the foregoing embodiments of the compound of formula (I), X is a bond, —(CH₂)ₒNR⁵—, or —NR⁵(CH₂)ₒ—. Preferably, X is a bond or —NH(CH₂)ₒ— (e.g., —NH(CH₂)—or —NH(CH₂)₂—).

In any of the foregoing embodiments of the compound of formula (I), m is 1.

In any of the foregoing embodiments of the compound of formula (I), n is 0, 1, or 2.

In any of the foregoing embodiments of the compound of formula (I), o is 1 or 2.

In any of the foregoing embodiments R⁵ and R⁶ are each H.

In one embodiment a PRMT5 inhibitor is provided for use in accordance with the present disclosure, wherein the inhibitor is a compound of formula (I):

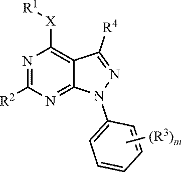

wherein
R¹ is selected from the group consisting of optionally substituted phenyl, piperazinyl, pyrrolyl, pyrrolidinyl pyranyl, piperidyl, morpholinyl, pyridinyl, and tetrahydrofuranyl;
R² is halo or —NH(CH₂)_{q}(N)CH₃CH₃;
R³ is H, C₁-C₈ alkyl or halo;
R⁴ is H;
X is —NH(CH₂)ₒ—;
o is 0, 1 or 2;
m is 0 or 1; and
q is 1 or 2, or a pharmaceutically acceptable salt thereof.
In one embodiment a compound of Formula I is provided wherein
R¹ is a substituted or di-substituted phenyl, wherein the phenyl substituents are independently selected from C₁-C₄ alkyl;
R² is halo or —NH(CH₂)_{q}(N)CH₃CH₃;
R³ is H or halo;
R⁴ is H;
X is —NH(CH₂)ₒ—;
o is 0, 1 or 2; and q is 2, optionally wherein the compound has the structure of

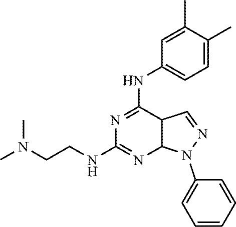 [SMILES: Cc1ccc(NC2=NC(NCCN(C)C)=NC3C2C=NN3c2ccccc2)cc1C]

Exemplary PRMT5 inhibitors for use in accordance with the present disclosure include compounds (Ia), (Ib), (Ic), (Id), (Ie), (If), and (Ig), as set forth below. Pharmaceutically acceptable salts of these exemplary compounds are also envisioned.

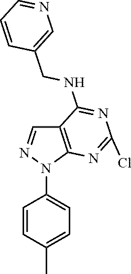 [SMILES: Cc1ccc(-n2ncc3c(NCc4cccnc4)nc(Cl)nc32)cc1]

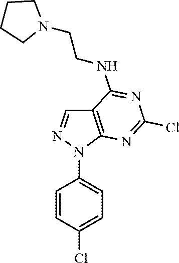 [SMILES: Clc1ccc(-n2ncc3c(NCCN4CCCC4)nc(Cl)nc32)cc1]

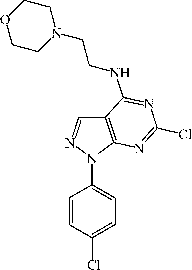 [SMILES: Clc1ccc(-n2ncc3c(NCCN4CCOCC4)nc(Cl)nc32)cc1]

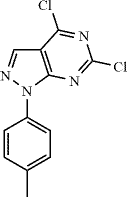 [SMILES: Cc1ccc(-n2ncc3c(Cl)nc(Cl)nc32)cc1]

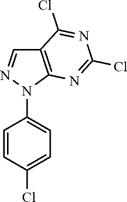 [SMILES: Clc1ccc(-n2ncc3c(Cl)nc(Cl)nc32)cc1]

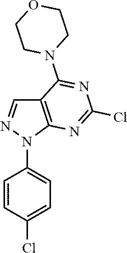 [SMILES: Clc1ccc(-n2ncc3c(N4CCOCC4)nc(Cl)nc32)cc1]

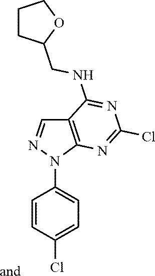

The present invention also provides a compound of formula (II):

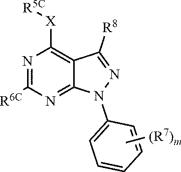

wherein
R^{5C} is halo, heterocycloalkyl, or heteroaryl, aryl or substituted aryl;
R^{6C} is C₁-C₈ alkyl, C₂-C₈ alkenyl, C₃-C₆ cycloalkyl, C₃-C₆ cycloalkylalkyl, heterocycloalkyl, aryl, arylalkyl, heteroaryl, C₁-C₈ alkoxy, C₃-C₆ cycloalkyloxy, aryloxy, halo, C₁-C₈ haloalkoxy, C₁-C₈ haloalkyl, haloaryl, haloaryloxy, —CN, —NO₂, —(CH₂)ₙC(O)R⁹, —(CH₂)ₙCO₂R⁹, —(CH₂)ₙC(O)NR⁹R¹⁰, —(CH₂)ₙNR⁹C(O) R¹⁰, —(CH₂)ₙNR⁹R¹⁰, —NR¹¹(CH₂)ₙNR⁹R¹⁰; or
two R^{6C} moieties and the phenyl group to which they are attached form a naphthyl group that is optionally substituted;
R⁷ is hydroxy, C₁-C₈ alkyl, C₂-C₈ alkenyl, C₃-C₆ cycloalkyl, C₃-C₆ cycloalkylalkyl, C₁-C₈ alkoxy, C₃-C₆ cycloalkyloxy, heterocycloalkyl, aryl, arylalkyl, heteroaryl, aryloxy, halo, C₁-C₈ haloalkyl, C₁-C₈ haloalkoxy, haloaryl, haloaryloxy, —CN, —NO₂, —C(O)R⁹, —CO₂R⁹, —C(O)NR⁹R¹⁰, —NR⁹C(O)R¹⁰, —(CH₂)ₙNR⁹R¹⁰,
—(CH₂)ₙSO₂NR⁹R¹⁰, —(CH₂)ₙSO₂R⁹, aryl; or
two R⁷ moieties and the phenyl group to which they are attached form a naphthyl group that is optionally substituted;
R⁸ is H, hydroxy, C₁-C₈ alkyl, C₂-C₈ alkenyl, C₃-C₆ cycloalkyl, C₁-C₈ haloalkyl, —CN, —NO₂, —(CH₂)ₙNR⁹R¹⁰, heterocycloalkyl, aryl, or heteroaryl;
X is a bond, —(CH₂)ₒCR⁹R¹⁰—, —CR⁹R¹⁰(CH₂)ₒ—, —(CH₂)ₒNR⁹—, —NR⁹(CH₂)ₒ—, —(CH₂)ₒO—, or —O(CH₂)ₒ—,
R⁹, R¹⁰, and R¹¹ are the same or different and each is H or C₁-C₈ alkyl;
m, n, and o are the same or different and each is 0 or an integer from 1-5, or
a pharmaceutically acceptable salt thereof.

In any of the foregoing embodiments of the compound of formula (II), R⁵ is aryl, optionally a substituted or di-substituted phenyl, wherein the substituents are selected from C₁-C₄ alkyl, R⁶ is —NR⁷(CH₂)ₙNR₅R₆, halo, C₁-C₈ haloalkoxy, C₁-C₈ haloalkyl, haloaryl, or haloaryloxy, optionally, R² is —NR⁷(CH₂)ₙNR⁹R¹⁰, R⁷ is H or halo, X is —(CH₂)ₒCR⁹R¹⁰ or —(CH₂)ₒNR⁹—, with R⁹ and R¹⁰, being independently H or C₁-C₈ alkyl, m is 1, o and n are independently 0 or 1 and R⁷ is H or halo.

In any of the foregoing embodiments of the compound of formula (II), R^{5C} is aryl, optionally a substituted or di-substituted phenyl, wherein the substituents are selected from C₁-C₄ alkyl; R^{6C} is —NR¹¹(CH₂)ₙNR⁹R¹⁰, halo, C₁-C₈ haloalkoxy, C₁-C₈ haloalkyl, haloaryl, or haloaryloxy; R⁷ is H, X is —(CH₂)ₒCR⁹R¹⁰, —NR⁹(CH₂)ₒ— or —(CH₂)ₒNR⁹—, with R⁹ and R¹⁰, being independently H or C₁-C₈ alkyl; m is 0, or 1; o and n are independently 0, 1, or 2; and R¹¹ is H or halo.

An exemplary compound of formula (II) is compound (IIa). Pharmaceutically acceptable salts of the exemplary compound are also envisioned.

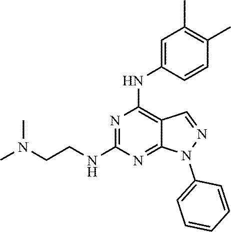

The present invention also provides a compound of formula (III):

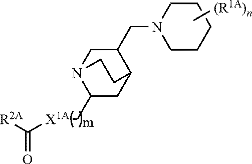

wherein
R^{1A} and R^{2A} are the same or different and each is hydrogen, C₁-C₈ alkyl, C₂-C₈ alkenyl, C₃-C₆ cycloalkyl, C₃-C₆ cycloalkylalkyl, heterocycloalkyl, aryl, arylalkyl, heteroaryl, hydroxy, C₁-C₈ alkoxy, C₃-C₆ cycloalkyloxy, aryloxy, halo, C₁-C₈ haloalkoxy, C₁-C₈ haloalkyl, haloaryl, haloaryloxy, —CN, —NO₂, —(CH₂)ₙC(O)R^{11A}, —(CH₂)ₙCO₂R^{11A}, —(CH₂)ₙC(O)NR^{11A}R^{12A}, —(CH₂)ₙNR^{11A}C(O)R^{12A}, or —(CH₂)ₙNR^{11A}R^{12A}; wherein each moiety other than hydrogen, hydroxy, halo, —CN, and —NO₂ is optionally substituted;
X^{1A} is O, S, or -NR^{11A};
R^{11A} and R^{12A} are the same or different and each is H or C₁-C₈ alkyl;
m is an integer from 1-4; and
n is 0 or an integer from 1-5, or
a pharmaceutically acceptable salt thereof.

An exemplary compound of formula (III) is compound (IIIa). Pharmaceutically acceptable salts of the exemplary compound also are envisioned.

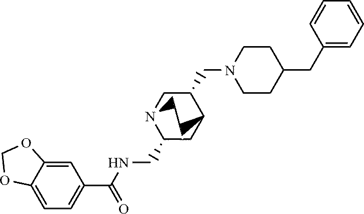 [SMILES: O=C(NC[C@H]1C[C@@H]2CC[N@@]1C[C@@H]2CN1CCC(Cc2ccccc2)CC1)c1ccc2c(c1)OCO2]

The present invention further provides a compound of formula (IV):

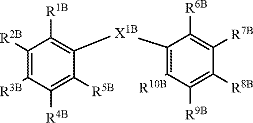

wherein
R^{1B}, R^{2B}, R^{3B}, R^{4B}, R^{5B}, R^{6B}, R^{7B}, R^{8B}, R^{9B}, and R^{10B} are the same or different and each is hydrogen, C₁-C₈ alkyl, C₂-C₈ alkenyl, C₃-C₆ cycloalkyl, C₃-C₆ cycloalkylalkyl, heterocycloalkyl, aryl, arylalkyl, heteroaryl, hydroxy, C₁-C₈ alkoxy, C₃-C₆ cycloalkyloxy, aryloxy,
haloaryloxy, —CN, —NO₂, halo, C₁-C₈ haloalkoxy, C₁-C₈ haloalkyl, haloaryl, —(CH₂)ₙC(O)R^{11B}, —(CH₂)ₙCO₂R^{11B}, —(CH₂)ₙC(O)NR^{11B}R^{12B}, —(CH₂)ₙNR^{11B}C(O)R^{12B}, or —(CH₂)ₙNR^{11B}R^{12B}; wherein each moiety other than hydrogen, hydroxy, halo, —CN, and —NO₂ is optionally substituted;
X^{1B} is O, S, or —NR^{11B}; and
R^{11B} and R^{12B} are the same or different and each is H or C₁-C₈ alkyl, or
a pharmaceutically acceptable salt thereof.

An exemplary compound of formula (IV) is compound (IVa).

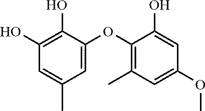 [SMILES: COc1cc(C)c(Oc2cc(C)cc(O)c2O)c(O)c1]

Pharmaceutically acceptable salts of the exemplary compound are also envisioned. It should be understood that the pharmaceutical compositions of the present disclosure can further include additional known therapeutic agents, drugs, modifications of the synthetic compounds into prodrugs, and the like for alleviating, mediating, preventing, and treating the diseases, disorders, and conditions described herein. For example, in one embodiment, the PRMT5 inhibitors can be administered with one or more anti-vascular endothelial growth factor (VEGF) agents, including, but not limited to, pegaptanib, ranibizumab, aflibercept, bevacizumab, brolucizumab (also known as ESBA1008 and RTH258), conbercept (also known as KH-902), abicipar pegol, pazopanib, regorafenib, and PAN-90806 and combinations thereof.

The pharmaceutical compositions including the PRMT5 inhibitors and, optionally, additional therapeutic agents and pharmaceutical carriers, used in the methods of the present disclosure can be administered to a subset of subjects in need of treatment for neovascular eye disease (a.k.a. ocular neovascular eye disease or ocular neovascular disease), including retinopathy of prematurity (ROP), proliferative diabetic retinopathy (PDR), wet age-related macular degeneration (AMD), neovascular age-related macular degeneration (nvAMD), pathological myopia, hypertensive retinopathy, occlusive vasculitis, polypoidal choroidal vasculopathy, diabetic macular edema, uveitic macular edema, central retinal vein occlusion, branch retinal vein occlusion, corneal neovascularization, retinal neovascularization, ocular histoplasmosis, neovascular glaucoma, retinoblastoma, and the like. Some subjects that are in specific need of treatment for ocular neovascular disease may include subjects who are susceptible to, or at elevated risk of, experiencing ocular neovascular disease (e.g., retinopathy of prematurity, diabetic retinopathy, “wet” age-related macular degeneration, neovascular age-related macular degeneration (nvAMD), etc.), and the like. Subjects may be susceptible to, or at elevated risk of, experiencing ocular neovascular diseases due to family history, age, environment, and/or lifestyle. Based on the foregoing, because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions.

### BIOLOGICAL EXAMPLES

### Example 1

The instant example provides exemplary materials and methods presented in Examples 2-20 described herein.

Animals. All animal studies were approved by the Institutional Animal Care and Use Committee, Indiana University School of Medicine and the “Use of Animals in Ophthalmic and Visual Research” guidelines of the Association for Research in Vision and Ophthalmology and ARRIVE guidelines were followed. Wild-type C57BL/6J mice (female, 7 weeks of age) were purchased from Jackson Laboratory (Bar Harbor, ME, USA) and housed under standard conditions at the Laboratory Animal Resource Center, Indiana University School of Medicine.

Laser-induced choroidal neovascularization (L-CNV) model. L-CNV was done as described before with minor modifications in laser power and duration. Briefly, mice were anesthetized by intraperitoneal injections of ketamine hydrochloride (80 mg/kg) and xylazine (10 mg/kg). The pupils of the eyes were dilated using tropicamide (1%) and phenylephrine (2.5%) and the eyes were exposed to laser treatment with 270 mW power pulses of the Micron IV laser injector (Phoenix Research Labs, Pleasanton, CA, USA) using 532 nm wavelength, 70 ms duration and 50 µm spot size. Mice were euthanized and eyes removed at days 1, 7 and 14. The untouched control mice underwent enucleation at the end of the experiment on day 14. Eyes were fixed in 4% paraformaldehyde (PFA).

Cell culture. The HRECs were purchased from Cell Systems (Kirkland, WA, USA) and grown in endothelial basal medium (EBM) in combination with endothelial cell growth medium (EGM-2) bullet kit (Lonza, Walkersville, MD, USA). iCEC2 cells were provided by Dr. Robert F. Mullins’ laboratory at the University of Iowa and grown in endothelial cell growth medium (R&D Systems, Minneapolis, MN, USA). iCEC2 cells contain a temperature-sensitive hypomorphic T antigen and actively proliferate at 33° C., which was used for stock maintenance, while growth slows at 37° C., which was used for functional analyses. All cells were tested for mycoplasma contamination regularly.

Statistical analyses. The data were analyzed using GraphPad Prism software (version 9.2.0). Unpaired Student’s t-test with Welch’s correction was used for comparing two means, while one-way ANOVA with Dunnett’s post hoc tests was used for comparing more than two means. Two-sided p values (p<0.05) were considered statistically significant.

### Example 2

### Proliferation Assays

HRECs and iCEC2 cells overexpressing PRMT5 and shPRMT5 knockdown cell lines were plated at 2 × 10⁴ cells/well in a 6-well plate. Cells were seeded in triplicate and counted on different days using a hemocytometer. For compound effects on proliferation, HRECs and iCEC2 cells were evaluated as described earlier. In brief, the cells were seeded in 96-well black plates with clear bottom at a density of 2.5 × 10³ with 100 µl of growth media and incubated for a day. After 24 h, 1 µl of PR5-LL-CM01 (0.1 nM to 100 µM) or vehicle DMSO (at 1% final concentration to the cells) was added, and the plates were incubated for 44 h. To each well of the plates, 11.1 µl of Alamar blue reagent was added and the readings were taken after 4 hours in a Synergy H1 plate reader (BioTek, Winooski, VT) with 560 nm (excitation) and 590 nm (emission) wavelengths. Using GraphPad Prism 9.0, G1₅₀ (growth inhibitory concentration) was calculated for PR5-LL-CM01.

### Example 3

### Immunostaining

Deidentified human donor eyes from nvAMD patients and aged controls were sourced from the National Disease Research Interchange (NDRI, PA, USA), which obtains informed consent for donor materials following the principles stated in the WMA Declaration of Helsinki and the Department of Health and Human services Belmont Report. The use of deidentified, decedent eyes was reviewed and designated “Not Human Subjects Research” by the Indiana University Institutional Review Board. The sections of the eye were deparaffinized, rehydrated and underwent antigen unmasking by heat-induced epitope retrieval in 1× citrate buffer (pH 6.0; Thermo Scientific, Waltham, MA, USA #AP-9003-500). The sections were rinsed with PBS and blocked for 2 hours at room temperature using 10% normal donkey serum (NDS; Abcam, #ab7475) prepared in 1% BSA in PBS, then incubated with primary (rabbit anti-PRMT5 at 1:100 dilution; Abcam, Waltham, MA, USA #ab109451; RRID:AB_10863428) and rabbit IgG control (1:100 dilution; R&D systems, Minneapolis, MN, USA # AB-105-C; RRID:AB_354266) antibodies prepared in 10% NDS plus 1% BSA for overnight at 4° C. Sections were washed thrice with PBS-T (PBS+0.025% Tween-20) for 5 minutes each. They were then incubated with Alexafluor 555-conjugated goat anti-rabbit secondary antibody (Invitrogen) at 1:200 dilution, for 1 hour in a dark humidified chamber at room temperature, followed by washing thrice with PBS-T for 5 minutes each. Finally, the sections were dehydrated through an ethanol series and mounted with Vectashield mounting medium with the nuclear stain, DAPI. Using 20× objective of a confocal microscope (LSM700, Carl Zeiss, Thomwood, NY, USA), the immunostained sections were imaged. The staining intensity of PRMT5 on these sections was quantified using ImageJ and mean fluorescence intensity (MFI) in the region of interest (ROI) was calculated by dividing the sum total of all the pixels by the number of pixels in order to rigorously assess if the nvAMD sections are expressing PRMT5 at levels contrasting to control sections analyzed.

The mouse eyes harvested after L-CNV induction at different days along with the eyes from untouched control mice were fixed in 4% PFA (Thermo Fisher Scientific, #43368) for 16 hours at 4° C. Dissected choroid and retina were embedded in optimal cutting temperature compound and cryosectioned to 5 µm thickness. The same immunostaining protocol described above was followed excluding deparaffinization, rehydration and antigen unmasking steps. The reagents used for mouse cryosections were as follows: primary rabbit anti-PRMT5/rabbit IgG control (1:150 dilution), GS-IB4 (1:250 dilution) and secondary Alexafluor 555-conjugated goat anti-rabbit antibody (1:250 dilution) and DyLight 488-conjugated streptavidin (1:400 dilution). The mounted mouse sections were imaged under the LSM700 confocal microscope with a 20× objective.

### Example 4

### Flat-Mount Staining

The RPE/choroids and retinas were dissected from the fixed eyes and prepared as flat mounts for PRMT5 and isolectin B4 (IB4) vasculature staining. In brief, the retina and choroid were fixed again with 4% PFA overnight at 4° C. The tissues were then washed with phosphate-buffered saline (PBS, 1X) twice for 10 minutes each, permeabilized in blocking buffer containing 5% bovine serum albumin (BSA) and 0.5% Triton X-100 in PBS for 70 minutes. After blocking, the tissues were stained with isolectin B4 from Griffonia simplicifolia (GS-IB4; biotin-conjugated, 1:250 dilution, Invitrogen, Waltham, MA, USA #121414) and anti-PRMT5 (1:150 dilution, Abcam, Waltham, MA, USA #ab109451; RRID:AB_10863428) prepared in antibody blocking solution containing 0.5% BSA (Fisher , Pittsburgh PA, USA #BP9703-100) and 0.5% Triton X-100 (Sigma-Aldrich, St. Louis, MO, USA #T9284) for 48-72 hours at 4° C. in a shaker. Four times for 15 min each, the tissues were washed with 1× PBS (Thermo Scientific, Waltham, MA, USA #10010-023) and incubated overnight in a shaker protected from light at 4° C. with respective detection reagents (DyLight 488-conjugated streptavidin at 1:400 dilution, Invitrogen #21832; and Alexafluor 555-conjugated goat anti-rabbit antibody at 1:250 dilution, Invitrogen #A21428; RRID:AB_2535849) prepared in antibody blocking solution. Then the tissues were washed with 1× PBS for four times, 15 minutes each and finally the immunostained tissues were flat mounted on glass slides and cover-slipped using Fluoromount-G (Southern Biotechnology, Birmingham, AL, USA). Until imaging, the flat mounts were protected from light and stored at 4° C. The immunostained L-CNV lesions were imaged by confocal microscope (LSM700, Carl Zeiss, Thomwood, NY, USA) using 20× objective and Z-stack optical sectioning.

### Example 5

### Immunoblotting for Tissues

Immunoblotting from the mouse eye lysates was performed. Briefly, retina and choroid tissues dissected out from the L-CNV and untouched control mice (four eyes) were lysed for 20 min on ice in radioimmunoprecipitation assay (RIPA) buffer (Sigma #R0278) with protease inhibitors (cOmplete mini, #04693159001, Roche, Indianapolis, IN, USA) and phosphatase inhibitors (PhosSTOP, #04906837001, Roche). The samples were homogenized and centrifuged at 12,000 g for 15 min at 4° C. Supernatants (lysates) were separated, and protein concentrations were determined using bicinchoninic acid (BCA) protein assay. Equal amounts of protein (25-35 µg) from retina and choroid samples were resolved by 10% SDS-PAGE and transferred onto polyvinylidene fluoride (PVDF) membranes (Millipore, Burlington, MA, USA). The antibodies against PRMT5 (rabbit, 1:1000 dilution, Abcam, Waltham, MA, USA #ab109451) and (β-actin (mouse, 1:1000 dilution, Sigma-Aldrich, #A5316; RRID:AB_476743) were used to detect the proteins and secondary antibodies were anti-rabbit IgG peroxidase conjugated (1:10,000, Rockland, Rockland, MD, USA, #611-1302; RRID:AB_219720) and anti-mouse IgG peroxidase conjugated (1:10,000, Rockland, #610-1302; RRID:AB_219656). All of the antibody dilutions were prepared in Tris-buffered saline containing 2.5% BSA. Amersham ECL prime immunoblotting detection reagents (#RPN2236, GE Healthcare, Chicago, IL, USA) were used to detect immunoreactive bands on a ChemiDoc MP imaging system (Bio-Rad, Hercules, CA, USA).

### Example 6

### Immunoblotting for Cells

HRECs and iCEC2 cells were pelleted post treatment with PR5-LL-CM01 (0, 1.5, 3, or 6 µM respectively for 24 hours) in phosphate buffered saline (PBS). Pellets were lysed with lysis buffer [10mM Tris-Cl pH 8.0, 1 mM EDTA, 1% Triton X-100, 0.1% sodium deoxycholate, 0.1% SDS (sodium dodecyl sulfate), 14 mM NaCl, 1 mM phenylmethylsulfonyl fluoride]. Protein concentration for each sample was determined using Protein Assay Reagent (Bio-Rad, Hercules, CA, USA). Equal protein concentrations were run on a 10% SDS-PAGE (polyacrylamide gel electrophoresis) gel and transferred to a polyvinylidene difluoride membrane (Thermo Fisher Scientific). Membranes were exposed to anti-p65, anti-p65me2, anti-flag, anti-PRMT5, and anti-actin and their respective secondary antibodies, mouse or rabbit respectively, based on manufacturer’s instructions. Protein signal was detected using enhanced chemiluminescence (ECL) reagent (PerkinElmer) and quantified where indicated using ImageJ. The antibodies used were obtained from commercial sources: Rabbit anti-p65 (at 1: 3,000 dilution; Santa-Cruz Biotech, Dallas, TX, USA, #SC-109), rabbit anti-p65me2 (customized antibody, Genscript, Piscataway, NJ), mouse anti-FLAG M2 (at 1:3,000 dilution; Millipore-Sigma, MO, USA, #F-1804), mouse anti-(β-actin (at 1: 5,000 dilution; Sigma-Aldrich, St. Louis, MO, USA, #A5316), rabbit anti-PRMT5 (at 1:3,000 dilution; Abcam, Waltham, MA, USA #ab109451), goat anti-rabbit IgG (H+L) secondary antibody, HRP (at 1:3,000 dilution; ThermoFisher Scientific, Waltham, MA, USA, # 31460), goat anti-mouse IgG (H+L) secondary antibody, HRP (at 1:3,000 dilution; ThermoFisher Scientific, Waltham, MA, USA, # 62-6520).

### Example 7

### Construction of Stable Cells

FLAG-tagged WT-PRMT5 cDNA or FLAG-tagged WT-p65 cDNA was amplified by reverse transcription from total mRNA derived from 293 cells. The sequences of WT-PRMT5 (NCBI sequence reference: NM_006109.3) and WT-p65 (NCBI sequence reference: NM-021975) were confirmed via DNA sequencing and then cloned into respective pLVX-IRES-puro vectors (https://www.takarabio.com/documents/Vector%20Documents/pLVX-IRES-Puro%20Vector%20Information.pdf). Lentiviral vectors expressing five different PRMT5-directed shRNAs (target set RHS4533-EG10419), and the universal negative control, pLKO.1 (RHS4080) were purchased from Open Biosystems (Dharmacon, Lafayette, CO, USA). To generate stable cells, the lentiviral plasmid containing the DNA of interest or shRNAs targeting PRMT5 exons were transfected into a 293T packaging cell line to produce viruses. HRECs or iCEC2 cells were then infected with these viruses and further selected with 1 µg/ml of puromycin, as the lentiviral vector construct includes a puromycin resistance gene. Expression of the respective constructs was confirmed using immunoblotting with specific antibodies. Cells were used for further experiments, with HRECs used between passages 5 and 7.

### Example 8

### NF-κB Luciferase Assay

The NF-κB luciferase lentiviral construct pLA-NFκBmCMV-luc-H4-puro⁴³, an NF-κB reporter lentiviral vector consists of a firefly luciferase reporter gene under the control of a minimal (m)CMV promoter and six NF-κB-responsive elements from the immunoglobulin light chain gene (kind gift from Peter Chumakov, Russian Academy of Sciences, Moscow, Russia) was introduced in the respective cells using Lipofectamine™ LTX Reagent and PLUS Reagents (Thermo Fisher Scientific). Luciferase activity was measured after 48 hours (with or without drug treatment) using Reporter Lysis Buffer kit (Promega, Madison, WI) per manufacturer’s instructions and a Synergy H4 plate reader.

### Example 9

### Quantitative Reverse-Transcription Polymerase Chain Reaction (qRT-PCR)

Following treatment with PR5-LL-CM01 for 24 hours, total RNA was isolated from the respective cells using TRIzol. First strand complementary DNA was generated using SuperScript III First-Strand Synthesis Kit (Invitrogen, Carlsbad, CA). GAPDH was selected as the housekeeping gene for normalization; each gene was run along with GAPDH, and the difference between threshold cycles (C_{T}) was designated as ΔC_{T}. ΔΔC_{T} is the difference between their respective controls. qPCR was executed using FastStart Universal SYBR Green kit (Roche, Indianapolis, IN). Primers were designed using the Primer Express 3.0 software (Thermo Fisher Scientific).

GAPDH-F CCATCACCATCTTCCAGGAGCG;
GAPDH-R AGAGATGATGACCCTTTTGGC; VEGFA-F TTGCCTT
GCTGCTCTACCTCCA; VEGFA-R GATGGCAGTAGCTGCGCTGATA;
TNFA-F TGGCCCAGGCAGTCAGA; TNFA-R GGTTTGCTACAACAT
GGGCTACA. VEGFR2-F CCGGCCTGTGAGTGTAAAAAC; VEGF
R2-R CGTCTGGTTGTCATCTGGGA.

### Example 10

### Determination of Pharmacokinetics (PK) Parameters

PK parameters of PR5-LL-CM01 were determined in NSG mice following a single I.P. dose administration of 20 mg/kg followed by blood collection using facial saphenous vein at 1, 2, 4, 8, and 24 h (Table 1). The plasma samples were then sent to the Clinical Pharmacology Analytic Core for analysis. A method to quantify PR5-LL-CM01 in plasma was developed using acetaminophen as the internal standard, liquid-liquid extraction, and HPLC-MS/MS (ABSciex 5500 Q-TRAP). The mass spectrometer utilized an electrospray ionization probe run in positive mode. The multiple reaction monitoring (MRM) Q1/Q3 (m/z) transitions for PR5-LL-CM01 and acetaminophen are 402.1/357.3 and 152.0/109.9, respectively. The lower limit of quantification is 0.1 ng/mL using 20 µL of plasma. Variability was minimized in the method by using plastic tubes instead of glass tubes, and methyl tert butyl ether instead of ethyl acetate, dichloroethane, or hexane:ethyl acetate. The mobile phase uses formic acid.

TABLE 1

Pharmacokinetic Data

| | |
|---|---|
| Dosage | 20 mg/kg |
| Route of Administration | I.P. |
| Cmax | 489 ng/mL (1.2 µM) |
| tmax | 1h |
| AUC_{0-∞} | 1,087 ng*mL⁻¹*h |
| t_{½} | 3.3 h |

TABLE 1 is a table showing the results of a pharmacokinetic (PK) assay of PR5-LL-CM01 showing values of Cₘₐₓ (maximum plasma concentration), tₘₐₓ (the time at which the Cₘₐₓ is observed), AUC_{0-∞} (area under the curve from time 0 extrapolated to infinite time), and t_{½} (half-life).

### Example 11

### HREC Migration Assay

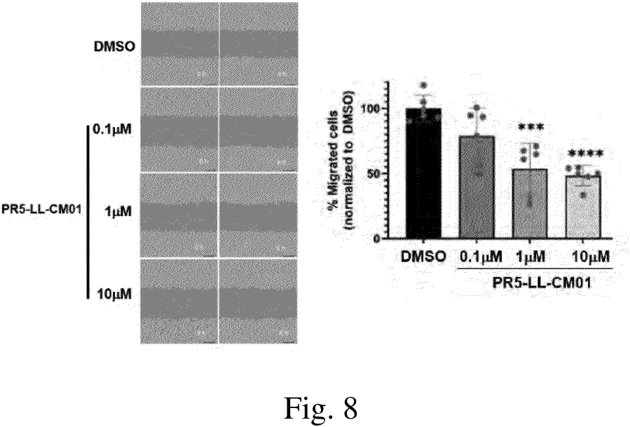

HRECs were grown until confluency in Essen ImageLock 96-well plates (cat no. 4379). The Essen WoundMaker was used to introduce a wound in the confluent growth and fresh complete medium containing DMSO or different concentrations of PR5-LL-CM01 was added to the wells (DMSO final concentration = 1%). Wells were imaged at 4x using an Incucyte ZOOM system (Essen Bioscience) with images taken every 2 hours to monitor cell migration across the scratch (FIG. 8). After 8 hours, the cells that migrated into the scratch were counted using the Multipoint tool in ImageJ. 6 images per treatment were normalized to DMSO control and analyzed by one-way ANOVA with Dunnett’s post hoc tests for comparisons between compounds’ treatments and control. All analysis was performed using GraphPad Prism software. A p-value of <0.05 was considered statistically significant.

### Example 12

### Matrigel Tube Formation Assay

The tube formation ability of HRECs, iCEC2 cells and the respective stable cells was assessed. Briefly, the wells of a 96 well plate were precoated with Matrigel (50 µl), which was allowed to solidify at 37° C. for 20 min. The cells were seeded at a density of 1.5 × 10⁴ in growth media (100 µl) containing 1 µl/well PR5-LL-CM01 (0.1 µM, 1 µM and 10 µM) or vehicle DMSO (1% final concentration to the cells). The stable cells were seeded at the same density over Matrigel basement membrane. Tube formation was monitored every 2 hours, after incubating the cells for 8 h at 37° C. and 5% CO₂, each well was photographed using a brightfield microscope (4× objective), and the measurements of formed tubes, meshes, branches, and segments were analyzed using AngiogenesisAnalyzer plugin in ImageJ software (v. 1.8.0; http://image.bio.methods.free.fr/ImageJ/?Angiogenesis-Analyzer-for-ImageJ.html).

### Example 13

### Flow Cytometry Cell Cycle Analysis

The analysis of cell cycle in HRECs and iCEC2 cells was assessed. Briefly, the cells were grown in 6 well plates (2 × 10⁶) and at 70% confluency, the cells were treated with PR5-LL-CM01 (0.1 µM, 1 µM and 10 µM) or vehicle DMSO (1% final concentration to the cells) for 24 h. The cells were washed with PBS twice and fixed in 70% ethanol. Prior to analysis, the cells were washed with PBS for two times, the cell pellets resuspended in propidium iodide (PI) staining solution (20 µg/ml) prepared in 1× PBS containing 100 µg/ml of RNAse A and 0.1% Triton X-100) and incubated at room temperature for 30 min. The cells were then analyzed on an Attune NxT flow cytometer (Thermo Fisher Scientific, Waltham, MA, USA). The single cell population was analyzed by area histograms and cell cycle profiles were created using Modfit software (v. 5.0, Verity Software House, Topsham, ME, USA). Pulse shape analysis was performed to eliminate any debris, doublets and aggregates from the whole cell population analyzed.

### Example 14

### PRMTS Inhibitor PR5-LL-CM01 Exhibits Antiangiogenic Properties in Ocular Endothelial Cells

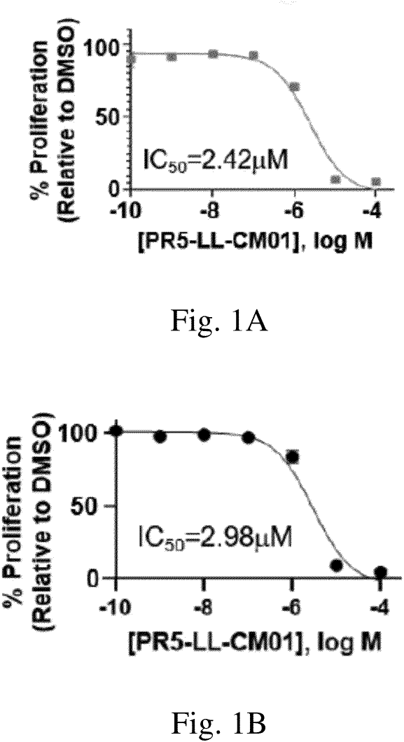

PR5-LL-CM01 has been identified as an effective PRMTS inhibitor which downregulates NF-κB activity. Previously, the effect of PR5-LL-CM01 has been shown only in the context of cancer. Here, we further studied PR5-LL-CM01 in the context of ocular angiogenesis, in HRECs and iCEC2 cells. Relative to DMSO, PR5-LL-CM01 dose-dependently reduced the proliferation of these cells in a low micromolar range, GI₅₀ = 2.42 µM in HRECs and 2.98 µM in iCEC2 cells (FIGS. 1A and 1B). Dose-dependent effects of PR5-LL-CM01 were observed. In vitro proliferation was measured using an alamarBlue assay. PR5-LL-CM01 is antiangiogenic in vitro. PR5-LL-CM01 is antiproliferative at 48 hours of treatment on HRECs (FIG. 1A) and on iCEC2 cells (FIG. 1B). Mean±SEM, n=3 technical replicates. Representative data from three biological replicates.

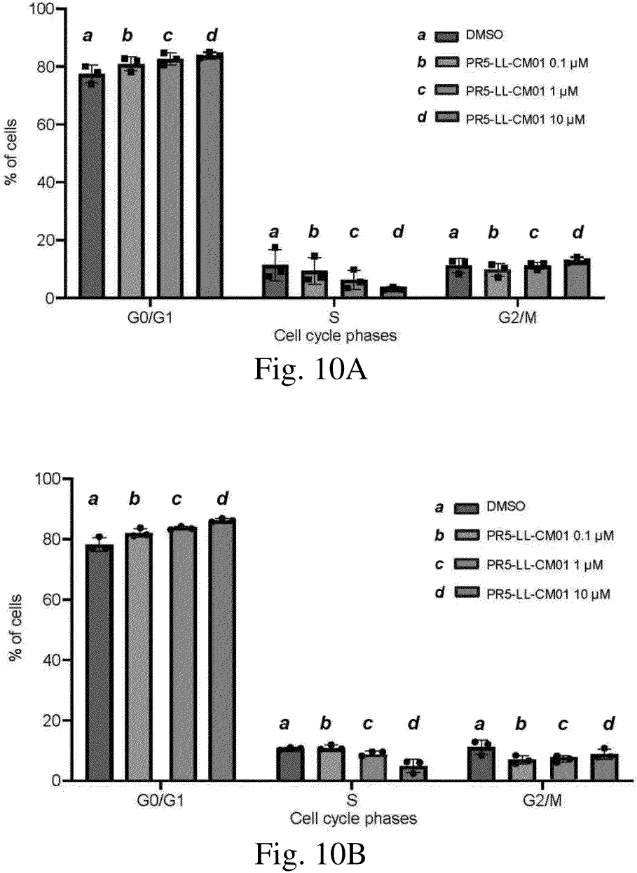

This antiproliferative effect was evident by the cell cycle arrest in these endothelial cells, where increasing concentrations of PR5-LL-CM01 decreased cells in S phase in comparison to the cells treated with 1% DMSO (FIGS. 10A and 10B). PR5-LL-CM01 dose-dependently decreases cells in S-phase and increases cells in G0/G1 after 24 hours of treatment in HRECs (FIG. 10A) and in iCEC2 cells (FIG. 10B). Mean±SEM of percentage of cells, n=3 biological replicates. Further, a concomitant increase in the cell populations was noted in the G0/G1 phase of the cell cycle on increasing doses of PR5-LL-CM01, suggesting a concentration-dependent inhibition of cell transition from G0/G1 to S-phase in both cell types.

### Example 15

### PRMTS Is Highly Expressed in nvAMD

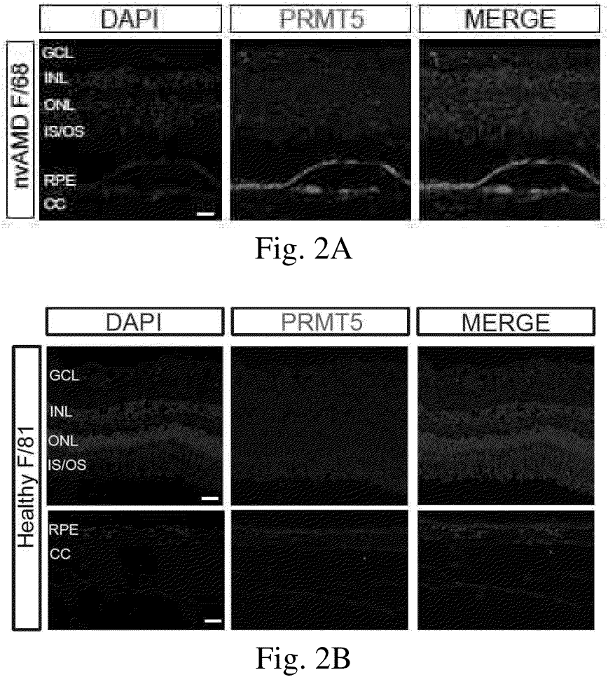

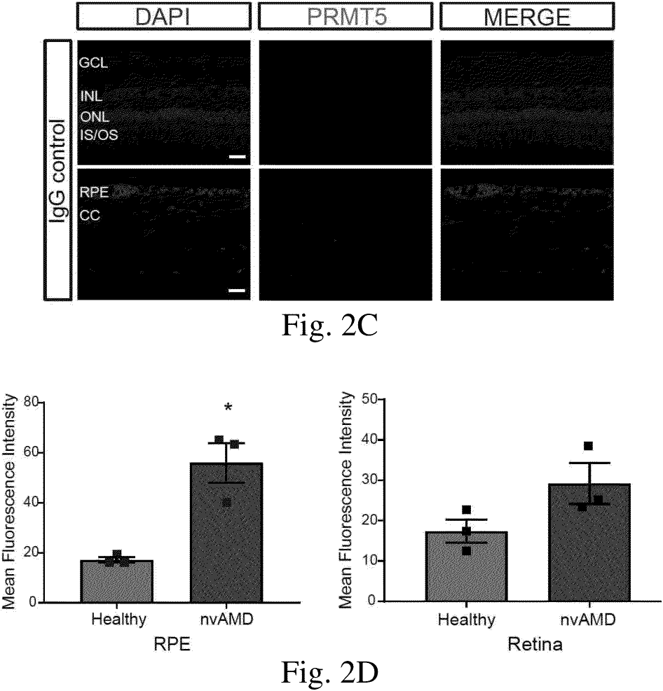

To investigate the potential role of PRMTS in ocular neovascularization, PRMTS expression in postmortem eyes from human nvAMD patients was examined. Sections of human nvAMD eyes in comparison with healthy control eyes showed PRMTS expression in all the layers of the retina, with especially high expression seen in the RPE-choroid where neovascularization originates in nvAMD (FIGS. 2A, 2B, and 2C). Quantification of staining intensity corroborated this observation that the intensity of PRMTS in RPE and retina was markedly higher in nvAMD samples than the healthy control samples (FIG. 2D). Also, AMD eyes compared to healthy control eyes clearly display the distinctive degenerative phenotypes, including the loss of inner of outer segments of photoreceptors and disruption in the retinal nuclear layer architecture (FIG. 2A)

### Example 16

### PRMTS Is Highly Expressed in Murine L-CNV

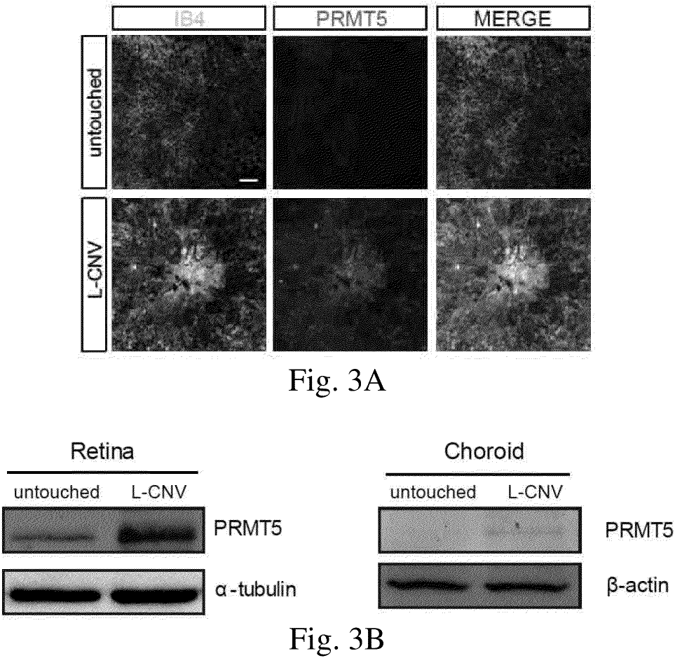

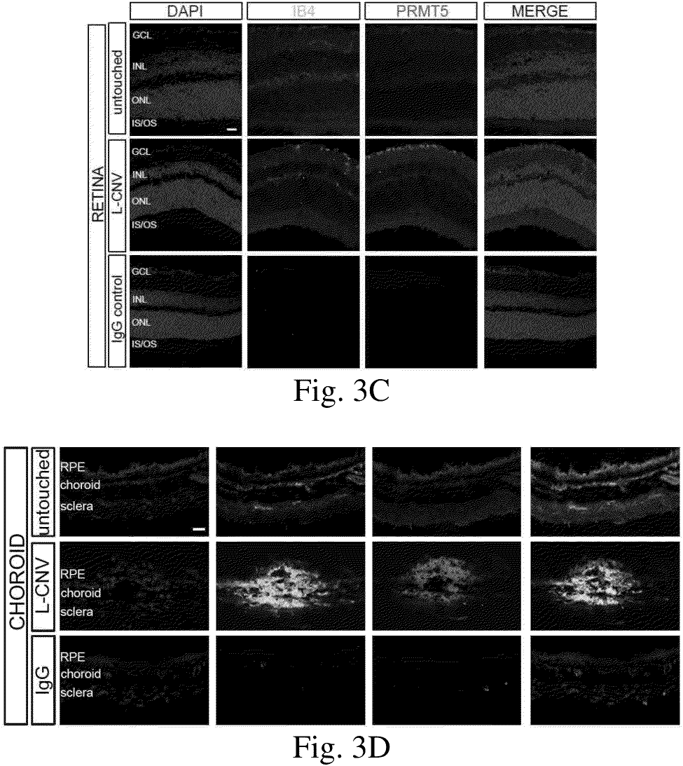

Given the expression of PRMTS in nvAMD, it was questioned whether PRMTS was similarly upregulated in experimental CNV. This was examined using a mouse model of L-CNV, wherein the mouse eyes were harvested at 7 days post laser treatment and assessed for the expression of PRMTS in the retina and choroid. Overexpression of PRMTS was observed in and around the L-CNV lesions in choroidal flat mounts compared to the flat mounts prepared from untouched control eyes (FIG. 3A). Immunoblot in comparison with untouched control eyes showed relatively increased levels of PRMTS both in retina and choroid (FIG. 3B) in the L-CNV samples. Immunostaining of PRMTS on mouse eye cryosections revealed high expression and localization of PRMTS in the ganglion cell layer (GCL) of the retina and in the RPE-Bruch’s membrane (BM)-choroid complex in L-CNV, although some expression was seen throughout the retina (FIGS. 3C and 3D).

### Example 17

### PRMTS Regulates Endothelial Cell Proliferation

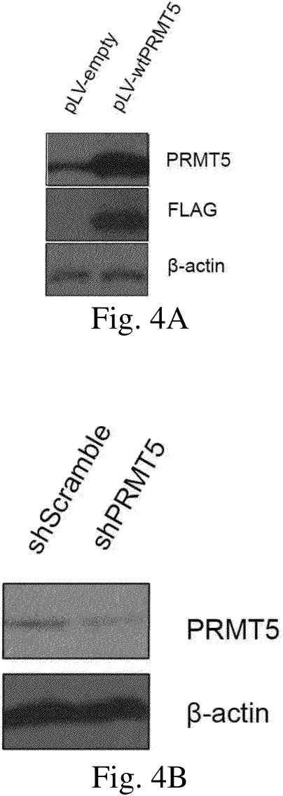

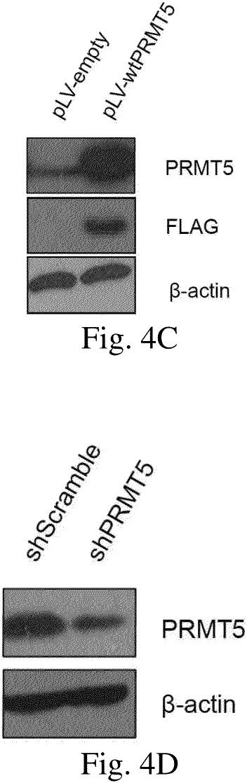

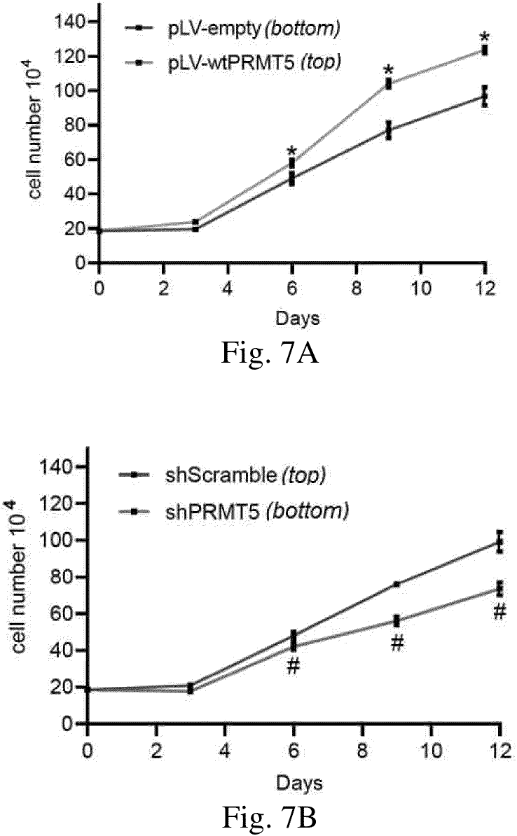

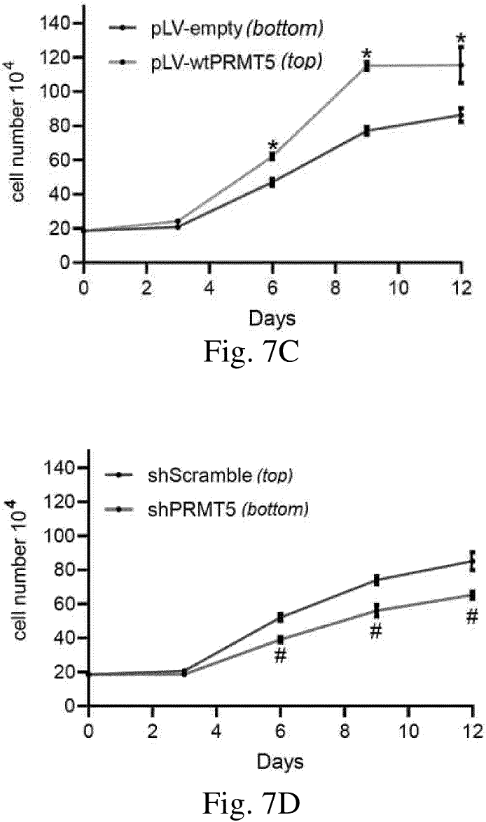

Due to the expression profile of PRMTS in CNV, PRMTS overexpression and shPRMTS knockdown in HRECs and iCEC2 cells was examined. Successful overexpression of a FLAG-tagged PRMTS protein in HRECs and iCEC2 cells compared to a vector control and to knock down PRMTS compared to shScramble controls was performed (FIGS. 4A to 4D). These cells were then used in future experiments. As NF-κB is an important regulator for cell growth, cellular proliferation assays were perfomed (FIGS. 7A to 7D). Overall, overexpression of PRMTS increased cellular growth compared to the vector control, while shRNA knockdown of PRMTS decreased growth compared to the shScramble control. These data may suggest PRMTS plays an important role in promoting ocular endothelial proliferation.

### Example 18

PR5-LL-CM01 decreases PRMTS-mediated NF-κB activation and its downstream target gene expression.

As PR5-LL-CM01 is known to inhibit NF-κB activity by downregulating PRMT5 activity in cancer, it was sought to determine if PR5-LL-CM01 has a direct effect on the dimethylation of the p65 subunit of NF-κB (p65me2), using p65 overexpressing HRECs and iCEC2 cells. Upon treatment with PR5-LL-CM01, p65me2 was decreased in a dose-dependent manner. This suggests PR5-LL-CM01 inhibited PRMTS-mediated p65me2.

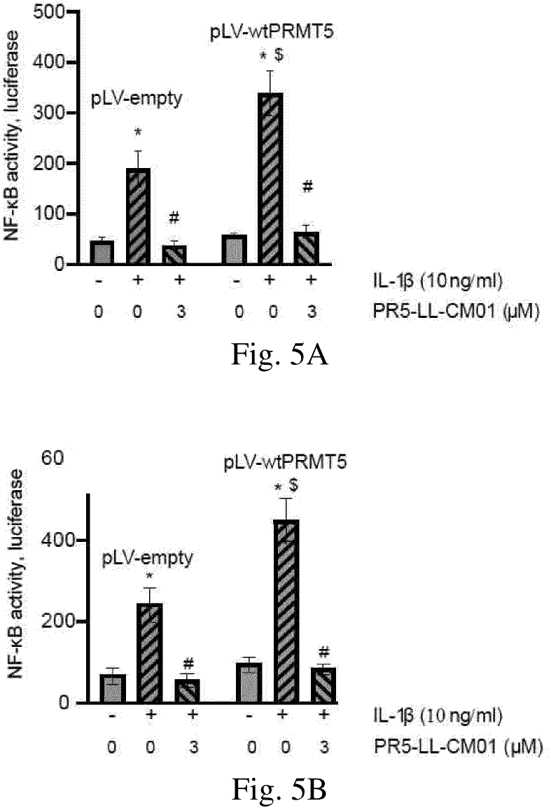

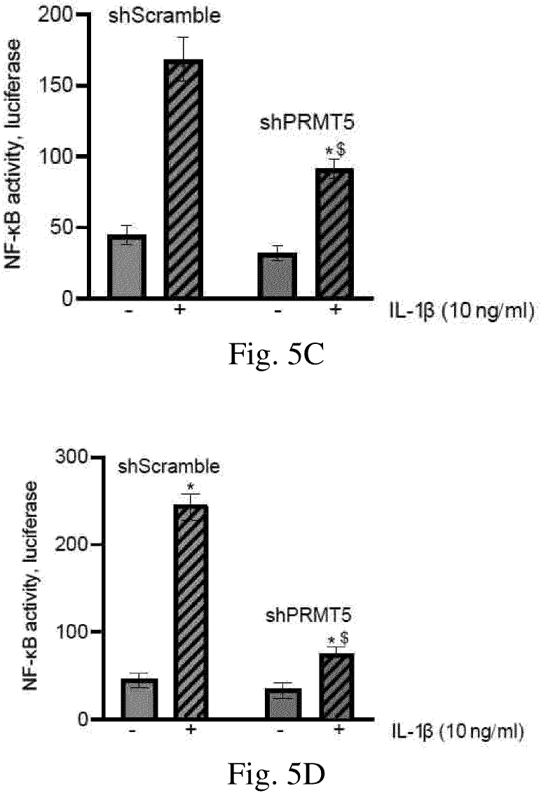

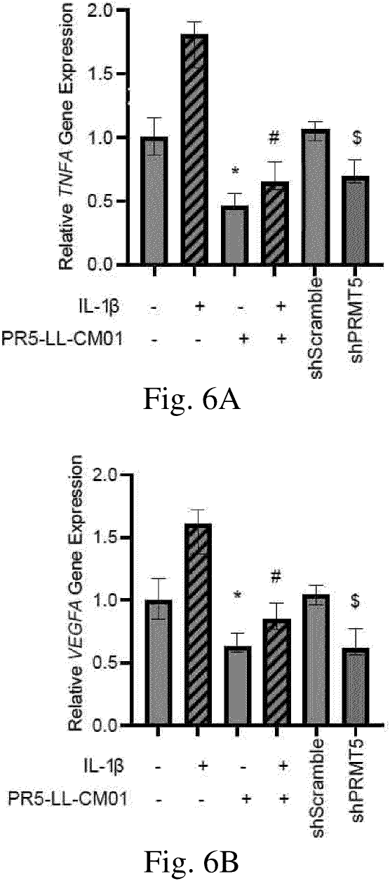

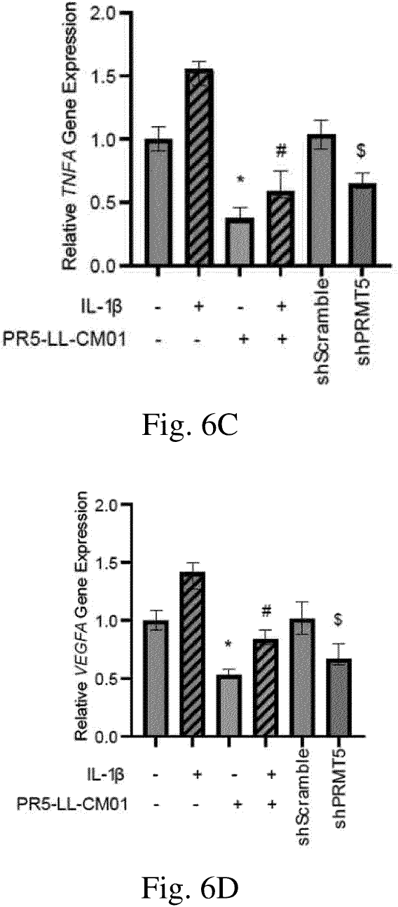

Given the documented regulation of NF-κB by PRMT5, it was sought to determine if PRMTS overexpression or shPRMTS knockdown altered NF-κB activity via a luciferase assay and if this activity could be reduced with the addition of PR5-LL-CM01 in retinal and choroidal endothelial cells. Overexpression of PRMTS significantly increased IL-1β-induced NF-κB activity, while addition of PR5-LL-CM01 dramatically reduced NF-κB activity in both HRECs (FIG. 5A) and iCEC2 cells (FIG. 5B). Furthermore, shPRMTS knockdown decreased IL-1 β-induced NF-κB activity compared to respective controls in both HRECs (FIG. 5C) and iCEC2 cells (FIG. 5D). To further evaluate the effect of PRMTS inhibition on NF-κB target gene expression, qPCR analysis was conducted of well-known NF-κB target genes involved in inflammation and angiogenesis: TNFA, VEGFA, and VEGFR2. The expression of TNFA and VEGFA (FIGS. 6A and 6B) were significantly decreased by either PR5-LL-CM01 treatment or shPRMTS knockdown as compared to their respective control cells, in both HRECs (FIG. 6A) and iCEC2 cells (FIG. 6B). To test for significant differences, an unpaired Student’s t-test with Welch’s correction was used for comparing two means, and one-way ANOVA with Dunnett’s post hoc tests was used when comparing more than two means.

Taken together, the above data may suggest that PR5-LL-CM01 decreases PRMT5-mediated NF-κB activation and its downstream target gene expression, thus presenting promising therapeutic antiangiogenic and anti-inflammatory potential through NF-κB inhibition.

### Example 19

### PRMTS Knockdown Reduces Angiogenic Tube Formation in Ocular Endothelial Cells

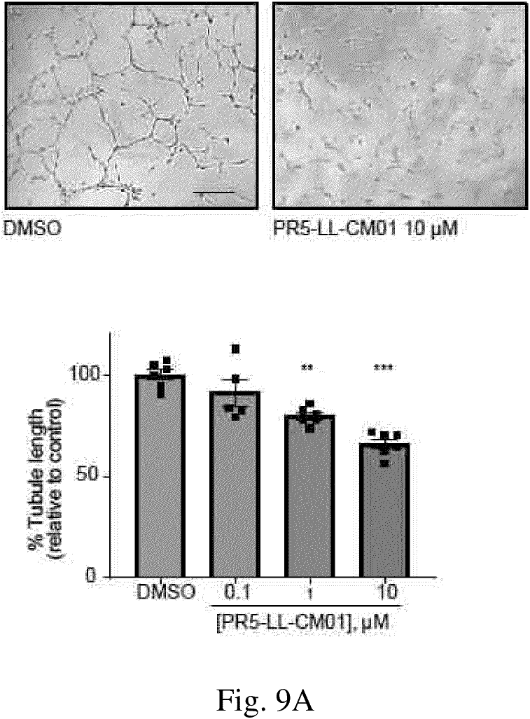

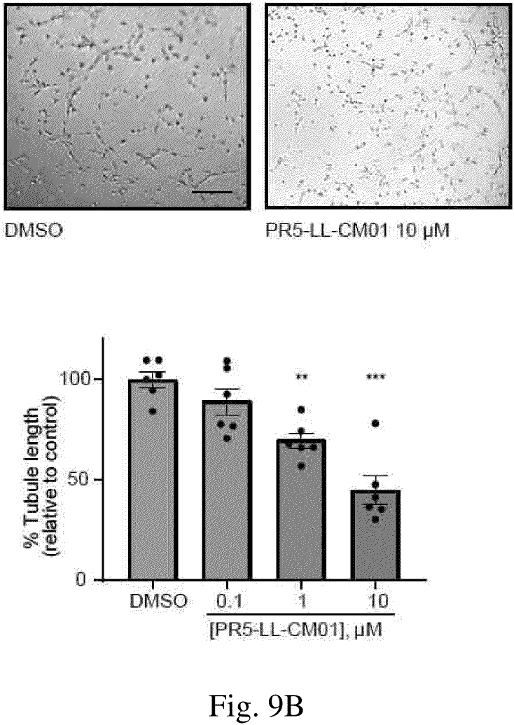

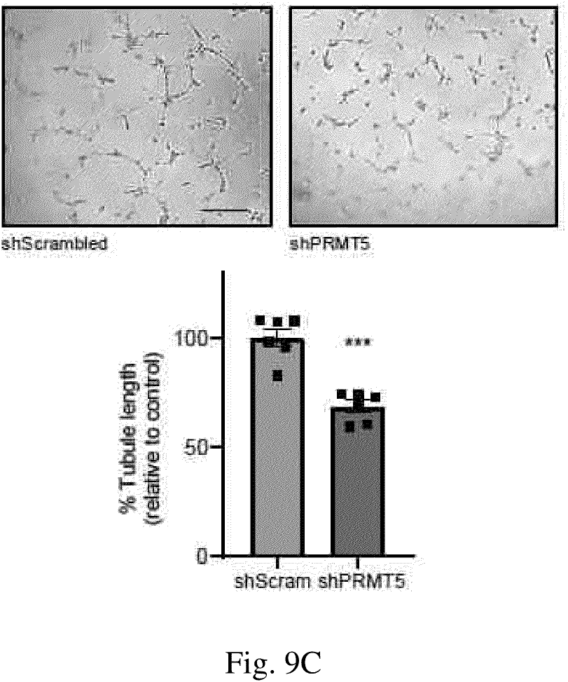

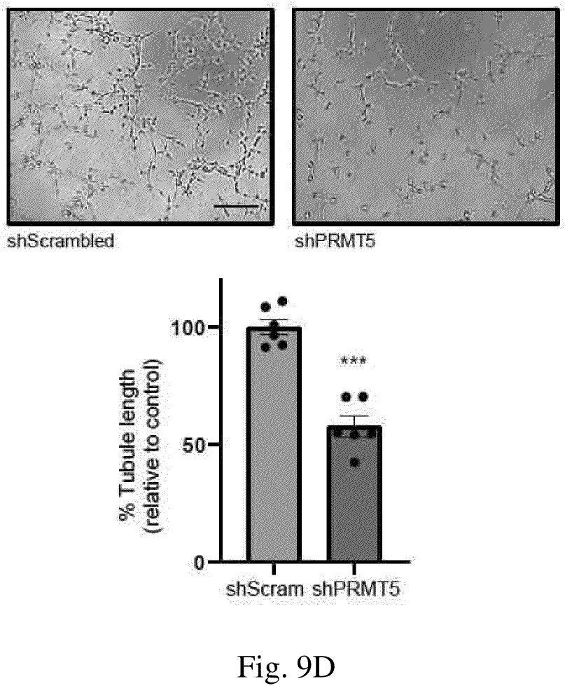

Tube formation is an in vitro property of endothelial cells reflective of angiogenic potential in vivo. The Matrigel tube formation assay is a widely used, reproducible model system to study either the activation or inhibition of angiogenic pathways in vitro. Knockdown of PRMTS in various cancer cells can suppress the protumorigenic functions of PRMT5. Therefore, it was sought to assess whether genetic or chemical inhibition of PRMTS affects tube formation in HRECs and iCEC2 cells. PRMTS inhibition by PR5-LL-CM01 dose-dependently decreased tube formation in both cell types (FIGS. 9A and 9B). Consistent with this, PRMTS knockdown (FIGS. 9C and 9D) in both cell types significantly reduced tube formation ability compared with the shScramble control. These data may suggest that inhibition of PRMTS by PR5-LL-CM01 is comparable with genetic knockdown and both can halt angiogenic properties of cells relevant to ocular neovascular diseases.

### Example 20

### Hypothetical Model

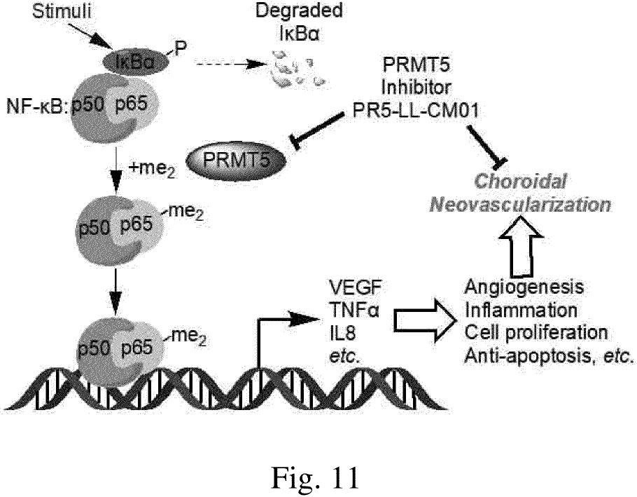

Based on the findings from the examples disclosed herein, evidence is suggested that PRMT5, an epigenetic enzyme, methylates and activates NF-κB in endothelial cells. This may result in the induction of NF-κB downstream genes, known to include cytokines, angiogenesis factors, chemokines, and antiapoptotic genes, whose functions are critical for inflammation and angiogenesis. Thus, using PR5-LL-CM01 to block the activity of PRMTS has potential to inhibit neovascularization-associated eye diseases (FIG. 11).

We claim:

1. A method of treating eye disease in a subject, said method comprising administering to the subject a therapeutically effective amount of an inhibitor of protein arginine methyltransferase 5 (PRMT5).

2. The method of claim 1, wherein the eye disease is a neovascular eye disease.

3. The method of claim 1, wherein the eye disease is retinopathy of prematurity (ROP), proliferative diabetic retinopathy (PDR), wet age-related macular degeneration (AMD), neovascular age-related macular degeneration (nvAMD), pathological myopia, hypertensive retinopathy, occlusive vasculitis, polypoidal choroidal vasculopathy, diabetic macular edema, uveitic macular edema, central retinal vein occlusion, branch retinal vein occlusion, corneal neovascularization, retinal neovascularization, ocular histoplasmosis, neovascular glaucoma, or retinoblastoma.

4. The method of claim 3, wherein the eye disease is neovascular age-related macular degeneration (nvAMD).

5. The method of claim 1, wherein the inhibitor decreases the amount of active PRMT5 present in the eye of said subject.

6. The method of claim 1, wherein the method comprises administering to a subject a PRMT5 inhibitor selected from the group consisting of compounds of formula (I), formula (II), formula (III), and formula (IV) 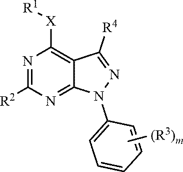 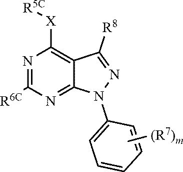 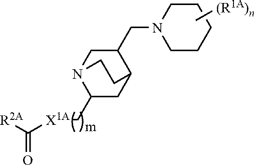 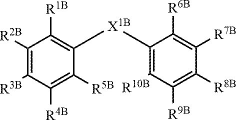
wherein
R¹ is hydrogen, halo, C₁-C₈ alkyl, C₃-C₆ cycloalkyl, heterocycloalkyl, aryl, or heteroaryl;
R² is C₁-C₈ alkyl, C₂-C₈ alkenyl, C₃-C₆ cycloalkyl, C₃-C₆ cycloalkylalkyl, heterocycloalkyl, aryl, arylalkyl, heteroaryl, C₁-C₈ alkoxy, C₃-C₆ cycloalkyloxy, aryloxy, halo, C₁-C₈ haloalkoxy, C₁-C₈ haloalkyl, haloaryl, haloaryloxy, —CN, —NO₂, —(CH₂)ₙC(O)R⁵, —(CH₂)ₙCO₂R⁵, —(CH₂)ₙC(O)NR⁵R⁶, —(CH₂)ₙNR⁵R⁶, —NH(CH₂)_{q}(N)CH₃CH₃, or -(CH₂)ₙNR⁵C(O)R⁶;
R³ is H, hydroxy, C₁-C₈ alkyl, C₂-C₈ alkenyl, C₃-C₆ cycloalkyl, C₃-C₆ cycloalkylalkyl, C₁-C₈ alkoxy, C₃-C₆ cycloalkyloxy, heterocycloalkyl, aryl, arylalkyl, heteroaryl, aryloxy, halo, C₁-C₈ haloalkyl, C₁-C₈ haloalkoxy, haloaryl, haloaryloxy, —CN, —NO₂, —C(O)R⁵, —CO₂R⁵, —C(O)NR⁵R⁶, —NR⁵C(O)R⁶, —(CH₂)ₙNR⁵R⁶, —(CH₂)ₙSO₂NR⁵R⁶, —(CH₂)ₙSO₂R⁵, aryl; or
two R³ moieties and the phenyl group to which they are attached form a naphthyl group that is optionally substituted;
R⁴ is H, hydroxy, C₁-C₈ alkyl, C₂-C₈ alkenyl, C₃-C₆ cycloalkyl, C₁-C₈ haloalkyl, —CN, —NO₂, —(CH₂)ₙNR⁵R⁶, heterocycloalkyl, aryl, or heteroaryl;
X is a bond, —(CH₂)ₒCR⁵R⁶—, —CR⁵R⁶(CH₂)ₒ—, —(CH₂)ₒNR⁵—, —NR⁵(CH₂)ₒ—, —(CH₂)ₒO—, or —O(CH₂)ₒ—,
R⁵ and R⁶ are the same or different and each is H or C₁-C₈ alkyl;
m, n, q and o are the same or different and each is 0 or an integer from 1-5, or a pharmaceutically acceptable salt thereof.

7. The method of claim 6, wherein the PRMT5 inhibitor is a compound selected from and 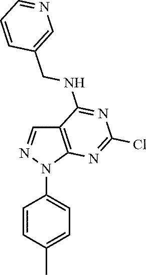 [SMILES: Cc1ccc(-n2ncc3c(NCc4cccnc4)nc(Cl)nc32)cc1] 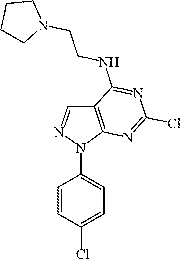 [SMILES: Clc1ccc(-n2ncc3c(NCCN4CCCC4)nc(Cl)nc32)cc1] 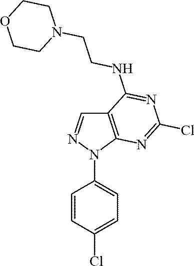 [SMILES: Clc1ccc(-n2ncc3c(NCCN4CCOCC4)nc(Cl)nc32)cc1] 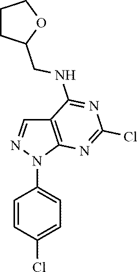 [SMILES: Clc1ccc(-n2ncc3c(NCC4CCCO4)nc(Cl)nc32)cc1]
or a pharmaceutically acceptable salt thereof.

8. The method of claim 6, wherein the PRMT5 inhibitor is a compound of formula (I): 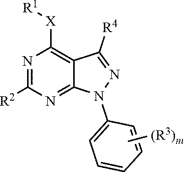
wherein
R¹ is selected from the group consisting of optionally substituted phenyl, piperazinyl, pyrrolyl, pyrrolidinyl pyranyl, piperidyl, morpholinyl, pyridinyl, and tetrahydrofuranyl;
R² is halo, —(CH₂)ₒCR⁵R⁶—, —(CH₂)ₒNR⁵ or —NH(CH₂)_{q}(N)R⁵R⁶;
R³ is H, C₁-C₈ alkyl or halo;
R⁴ is H or halo;
R⁵ and R⁶ are independently H or C₁-C₄ alkyl;
X is —NH(CH₂)ₒ—;
o is 0, 1 or 2;
m is 0 or 1; and
q is 1 or 2, or a pharmaceutically acceptable salt thereof.

9. The method of claim 8, wherein the PRMT5 inhibitor has the structure of 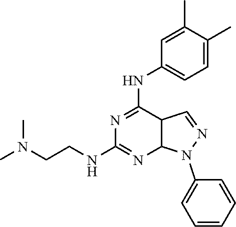 [SMILES: Cc1ccc(NC2=NC(NCCN(C)C)=NC3C2C=NN3c2ccccc2)cc1C]
or a pharmaceutically acceptable salt thereof.

10. The method of claim 1, wherein the PRMT5 inhibitor is administered via eye drops, eye ointment, intravitreal injection, orally, or any combination thereof.

11. The method of claim 1, further comprising administering to the subject a therapeutically effective amount of a VEGF inhibitor.

12. The method of claim 11, wherein the VEGF inhibitor is selected from the group comprising pegaptanib, ranibizumab, aflibercept, bevacizumab, brolucizumab (also known as ESBA1008 and RTH258), conbercept (also known as KH-902), abicipar pegol, regorafenib, PAN-90806, Votrient (Generic name: pazopanib), Sutent (Generic name: sunitinib), Avastin (Generic name: bevacizumab), Nexavar (Generic name: sorafenib), Stivarga (Generic name: regorafenib), Cabometyx (Generic name: cabozantinib), Lenvima (Generic name: lenvatinib), Iclusig (Generic name: ponatinib), Cometriq (Generic name: cabozantinib), Zaltrap (Generic name: ziv-aflibercept), Inlyta (Generic name: axitinib), Zirabev (Generic name: bevacizumab), Mvasi (Generic name: bevacizumab), Fotivda (Generic name: tivozanib), Cyramza (Generic name: ramucirumab), and Caprelsa (Generic name: vandetanib).

13. A method of inhibiting retinal angiogenesis, said method comprising contacting retinal endothelial cells with an inhibitor of protein arginine methyltransferase 5 (PRMT5).

14. The method of claim 13, said method comprising contacting retinal endothelial cells with a PRMT5 inhibitor having a structure selected from the group consisting of formula (I), formula (II), formula (III), and formula (IV) 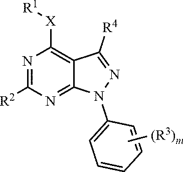 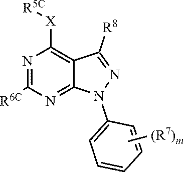 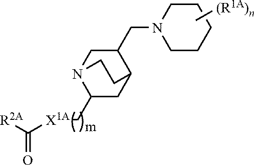 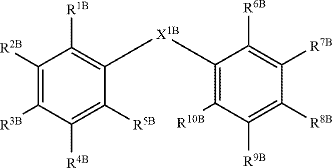
wherein
R¹ is hydrogen, halo, C₁-C₈ alkyl, C₃-C₆ cycloalkyl, heterocycloalkyl, aryl, or heteroaryl;
R² is C₁-C₈ alkyl, C₂-C₈ alkenyl, C₃-C₆ cycloalkyl, C₃-C₆ cycloalkylalkyl, heterocycloalkyl, aryl, arylalkyl, heteroaryl, C₁-C₈ alkoxy, C₃-C₆ cycloalkyloxy, aryloxy, halo, C₁-C₈ haloalkoxy, C₁-C₈ haloalkyl, haloaryl, haloaryloxy, —CN, —NO₂, —(CH₂)ₙC(O)R⁵, —(CH₂)ₙCO₂R⁵, —(CH₂)ₙC(O)NR⁵R⁶, —(CH₂)ₙNR⁵R⁶, —NH(CH₂)_{q}(N)CH₃CH₃, or —(CH₂)ₙNR⁵C(O)R⁶_{;}
R³ is H, hydroxy, C₁-C₈ alkyl, C₂-C₈ alkenyl, C₃-C₆ cycloalkyl, C₃-C₆ cycloalkylalkyl, C₁-C₈ alkoxy, C₃-C₆ cycloalkyloxy, heterocycloalkyl, aryl, arylalkyl, heteroaryl, aryloxy, halo, C₁-C₈ haloalkyl, C₁-C₈ haloalkoxy, haloaryl, haloaryloxy, —CN, —NO₂, —C(O)R⁵, —CO₂R⁵, —C(O)NR⁵R⁶, —NR⁵C(O)R⁶, —(CH₂)ₙNR⁵R⁶, —(CH₂)ₙSO₂NR⁵R⁶, —(CH₂)ₙSO₂R⁵, aryl; or
two R³ moieties and the phenyl group to which they are attached form a naphthyl group that is optionally substituted;
R⁴ is H, hydroxy, C₁-C₈ alkyl, C₂-C₈ alkenyl, C₃-C₆ cycloalkyl, C₁-C₈ haloalkyl, —CN, —NO₂, —(CH₂)ₙNR⁵R⁶, heterocycloalkyl, aryl, or heteroaryl;
X is a bond, —(CH₂)ₒCR⁵R⁶—, —CR⁵R⁶(CH₂)ₒ—, —(CH₂)ₒNR⁵—, —NR⁵(CH₂)ₒ—, —(CH₂)ₒO—, or —O(CH₂)ₒ—,
R⁵ and R⁶ are the same or different and each is H or C₁-C₈ alkyl;
m, n, q and o are the same or different and each is 0 or an integer from 1-5, or a pharmaceutically acceptable salt thereof.

15. The method of claim 14, wherein the PRMT5 inhibitor is a compound selected from and 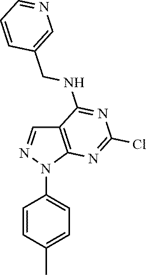 [SMILES: Cc1ccc(-n2ncc3c(NCc4cccnc4)nc(Cl)nc32)cc1] 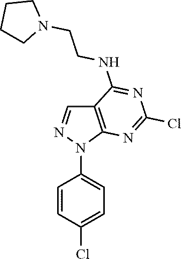 [SMILES: Clc1ccc(-n2ncc3c(NCCN4CCCC4)nc(Cl)nc32)cc1] 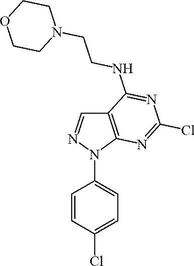 [SMILES: Clc1ccc(-n2ncc3c(NCCN4CCOCC4)nc(Cl)nc32)cc1] 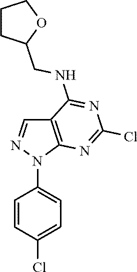 [SMILES: Clc1ccc(-n2ncc3c(NCC4CCCO4)nc(Cl)nc32)cc1]
or a pharmaceutically acceptable salt thereof.

16. The method of claim 14, wherein the PRMT5 inhibitor has the structure of 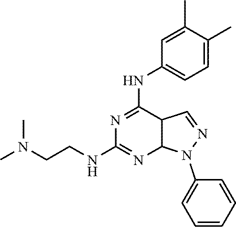 [SMILES: Cc1ccc(NC2=NC(NCCN(C)C)=NC3C2C=NN3c2ccccc2)cc1C]
or a pharmaceutically acceptable salt thereof.

17. A method of inhibiting choroidal angiogenesis, said method comprising contacting choroidal endothelial cells with an inhibitor of protein arginine methyltransferase 5 (PRMT5).

18. The method of claim 17, said method comprising contacting choroidal endothelial cells with a PRMT5 inhibitor having a structure selected from the group consisting of formula (I), formula (II), formula (III), and formula (IV) 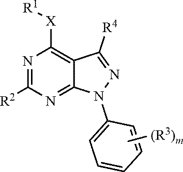 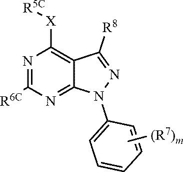 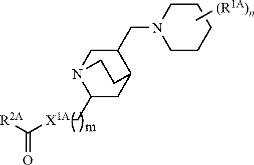 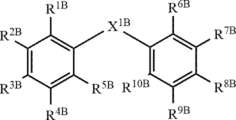
wherein
R¹ is hydrogen, halo, C₁-C₈ alkyl, C₃-C₆ cycloalkyl, heterocycloalkyl, aryl, or heteroaryl;
R² is C₁-C₈ alkyl, C₂-C₈ alkenyl, C₃-C₆ cycloalkyl, C₃-C₆ cycloalkylalkyl, heterocycloalkyl, aryl, arylalkyl, heteroaryl, C₁-C₈ alkoxy, C₃-C₆ cycloalkyloxy, aryloxy, halo, C₁-C₈ haloalkoxy, C₁-C₈ haloalkyl, haloaryl, haloaryloxy, —CN, —NO₂, —(CH₂)ₙC(O)R⁵, —(CH₂)ₙCO₂R⁵, —(CH₂)ₙC(O)NR⁵R⁶, —(CH₂)ₙNR⁵R⁶, —NH(CH₂)_{q}(N)CH₃CH₃, or —(CH₂)ₙNR⁵C(O)R⁶_{;}
R³ is H, hydroxy, C₁-C₈ alkyl, C₂-C₈ alkenyl, C₃-C₆ cycloalkyl, C₃-C₆ cycloalkylalkyl, C₁-C₈ alkoxy, C₃-C₆ cycloalkyloxy, heterocycloalkyl, aryl, arylalkyl, heteroaryl, aryloxy, halo, C₁-C₈ haloalkyl, C₁-C₈ haloalkoxy, haloaryl, haloaryloxy, —CN, —NO₂, —C(O)R⁵, —CO₂R⁵, —C(O)NR⁵R⁶, —NR⁵C(O)R⁶, —(CH₂)ₙNR⁵R⁶, —(CH₂)ₙSO₂NR⁵R⁶, —(CH₂)ₙSO₂R⁵, aryl; or
two R³ moieties and the phenyl group to which they are attached form a naphthyl group that is optionally substituted;
R⁴ is H, hydroxy, C₁-C₈ alkyl, C₂-C₈ alkenyl, C₃-C₆ cycloalkyl, C₁-C₈ haloalkyl, —CN, —NO₂, —(CH₂)ₙNR⁵R⁶, heterocycloalkyl, aryl, or heteroaryl;
X is a bond, —(CH₂)ₒCR⁵R⁶—, —CR⁵R⁶(CH₂)ₒ—, —(CH₂)ₒNR⁵—, —NR^{S}(CH₂)ₒ—, —(CH₂)ₒO—, or —O(CH₂)ₒ—,
R⁵ and R⁶ are the same or different and each is H or C₁-C₈ alkyl;
m, n, q and o are the same or different and each is 0 or an integer from 1-5, or a pharmaceutically acceptable salt thereof.

19. The method of claim 18, wherein the PRMT5 inhibitor is a compound selected from and 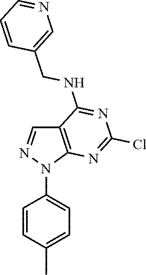 [SMILES: Cc1ccc(-n2ncc3c(NCc4cccnc4)nc(Cl)nc32)cc1] 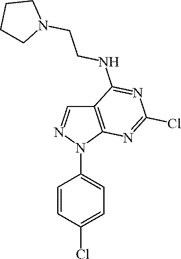 [SMILES: Clc1ccc(-n2ncc3c(NCCN4CCCC4)nc(Cl)nc32)cc1] 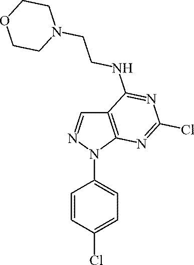 [SMILES: Clc1ccc(-n2ncc3c(NCCN4CCOCC4)nc(Cl)nc32)cc1]
and 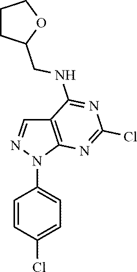 [SMILES: Clc1ccc(-n2ncc3c(NCC4CCCO4)nc(Cl)nc32)cc1]
or a pharmaceutically acceptable salt thereof.

20. The method of claim 18, wherein the PRMT5 inhibitor has the structure of 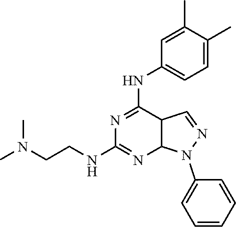 [SMILES: Cc1ccc(NC2=NC(NCCN(C)C)=NC3C2C=NN3c2ccccc2)cc1C]
or a pharmaceutically acceptable salt thereof.
